(19) United States
(12) Patent Application Publication
(10) Pub. No.: US 2018/0223350 A1
(43) Pub. Date: Aug. 9, 2018

(54) DUPLEX ADAPTERS AND DUPLEX SEQUENCING

(71) Applicant: Integrated DNA Technologies, Inc., Coralville, IA (US)

(72) Inventors: Brendan Galvin, Menlo Park, CA (US); Jiashi Wang, Redwood City, CA (US)

(73) Assignee: Integrated DNA Technologies, Inc., Coralville, IA (US)

(21) Appl. No.: 15/891,002

(22) Filed: Feb. 7, 2018

Related U.S. Application Data
(60) Provisional application No. 62/456,334, filed on Feb. 8, 2017.

(51) Int. Cl. C12Q 1/6855; C12Q 1/6869; C12N 15/10; C12Q 1/6876; C12N 15/11

(52) U.S. Cl. CPC C12Q 1/6855; C12Q 1/6869; C12N 2310/531; C12Q 1/6876; C12N 15/11; C12N 15/1065

(57) ABSTRACT

This invention pertains to the creation of a complex pool of adapters that contain complementary barcodes to be utilized in next generation sequencing library prep methods and methods of using barcoded adapters for next generation sequencing.

### CROSS-REFERENCE TO RELATED APPLICATIONS

This application claims benefit of priority under 35 U.S.C. 119 to U.S. provisional patent application bearing Ser. No. 62/456,334, filed Feb. 8, 2017, and entitled “LOOPED DUPLEX ADAPTERS AND DUPLEX SEQUENCING,” the contents of which are herein incorporated by reference in their entirety.

### SEQUENCE LISTING

The instant application contains a Sequence Listing that has been submitted in ASCII format via EFS-Web and is hereby incorporated by reference in its entirety. The ASCII copy, created Feb. 6, 2018, is named Sequence Listing.txt, and is 68,472 bytes in size.

### FIELD OF THE INVENTION

This invention pertains to the synthesis of individual non-degenerate and degenerate oligonucleotide adapters and looped duplex sequencing adapter sequences. Additionally, the invention pertains to methods for ligating duplex adapters and ligating looped duplex adapters for next generation sequencing target preparation.

### BACKGROUND OF THE INVENTION

Massively parallel DNA sequencing, or next generation sequencing (NGS), has allowed the sequencing of billions of bases in a small fraction of time. NGS has evolved into a very powerful tool in molecular biology, allowing for the rapid progress in fields such as genomic identification, genetic testing, drug discovery, and disease diagnosis. As this technology continues to advance, the volume of nucleic acids which can be sequenced at one time is increasing. This allows researchers to not only sequence larger samples, but to increase the number of reads per sample which allows for detection of small sequence variations within the sample.

As the volume and complexity of NGS process increases, so does the rate of experimental error. While much of this error occurs in the sequencing steps, error can also occur during sample preparation. This is particularly true during the conversion of the sample into a readable NGS library by which adapter sequences are attached to the ends of each fragment of a fragmented sample (library fragment) in a uniform fashion. This experimental error makes it difficult to detect rare mutations. Additionally, this experimental error makes it difficult to detect rare mutations in samples from cfDNA, liquid biopsies, FFPE DNA, or any sample where target material is limited.

Traditionally, NGS platforms generate sequence data from a single strand of DNA. In theory, DNA subpopulations of any size should be detectable when deep sequencing a large number of molecules. However, the inherent error rate of polymerases, which create point mutations from base misincorporation and rearrangement due to template switching (sometimes referred to as UMI hopping or jumping PCR) can result in incorrect mutation calls. Additionally, errors arise due to damage introduced to the template during NGS sample preparation. This combination of inherent polymerase error and sample preparation errors can result in incorrect variant calls. This is especially true when the mutation is present at extremely low frequency in a highly heterogeneous sample population. It is estimated that the error rate varies from about 0.06% to 1% depending on various factors which include read length, base calling, algorithms and the type of variants detected (see Kinde et al., Proc. Nat'l. Acad. Sci. U.S.A. 108:9530-5, 2011). Therefore, detecting true mutations below this background error rate is difficult without additional error correcting methods.

Amplification of target nucleic acid prior to or during sequencing by PCR may introduce artifactual errors. Additionally, DNA templates damaged during library preparation may be amplified and incorrectly categorized as mutations. A common approach to reduce or eliminate artifactual mutations arising from DNA damage, PCR errors, and sequencing errors involves tagging the starting molecule with unique molecular identifier tags (also known as molecular barcodes). These barcodes enable the precise tracking of individual molecules, making it possible to distinguish authentic somatic mutations arising in vivo from artifacts introduced ex vivo. These tags can be appended to a single strand of duplexed DNA molecule. To further increase the sensitivity of NGS unique molecular identifier tags are added to both strands of a duplexed DNA molecule. Tagging both strands of a duplexed DNA molecule thus further reduces errors. Because the two strands are complementary, true mutations are found at the same position in both strands, while polymerase introduced errors or sample preparation errors will likely occur in only one strand and the chances of an error occurring at the same position on both strands is extremely unlikely.

Efforts have been made to develop NGS-based rare variant detection. This is particularly true in cancer where genetic heterogeneity is common or there are multiple metastases. There exist three main barriers that limit the ability of NGS application to detect rare mutants or rare variants. These are the intrinsic error frequency of the NGS system, the number of reads a sequencing platform can produce and the amount of input DNA available.

The theoretical limit of detection (LOD) for detecting true mutants can broadly be given as the error rate post-duplex sequencing. This LOD has been reported to be between 10e-7 and 10e-6. However, achieving this level of sensitivity is often difficult or impractical due to the required target material needed and/or the sequencing depth required to at that level.

Prior methods rely on a two-part synthesis method to generate a partially double stranded barcoded adapter. A first oligonucleotide containing a barcode sequence is synthesized. The second strand, which is partially complementary to the fully barcoded adapter is subsequently synthesized. To generate a fully double stranded adapter the partial secondary strand is annealed to the first oligonucleotide and is then extended and filled in with a polymerase. This polymerase fill in creates a fully double stranded bar code region. However, polymerases do not replicate DNA sequences with 100% accuracy and can therefore introduce errors into the sequencing barcodes. The intrinsic error frequency of the polymerase used to fill in the adapter further reduce the accuracy and sensitivity for detecting rare mutants in NGS reactions.

Although the use of duplexed adapters having unique molecular identifiers has increased the sensitivity of NGS there is the is a need in the art for tag-based error correction methods that further reduce or eliminate artifactual mutations arising from DNA damage, polymerase errors, PCR errors, and sequencing errors. The ability to detect mutant population of a smaller and smaller size in a mixed population pool which is predominately wild type is needed. Methods and compositions for reducing or eliminating artifactual mutations would be useful in NGS applications, including, but not limited to, rare mutation detection, use in sequencing cfDNA, use in sequencing FFPE samples, use in single cell sequencing, or use in sequencing liquid biopsies or ctDNA.

### BRIEF SUMMARY OF THE INVENTION

The invention provides compositions comprising a complex pool of adapters containing complementary barcodes. Further the invention provides individually synthesized duplex barcoded adapters. Additionally, the invention includes methods for tagging a nucleic acid fragment for next generation sequencing library prep and sequencing.

Aspects of the present invention include methods of individually synthesizing oligonucleotides that contain barcodes and sequencing using the duplexed adapters including the steps of: annealing the individually synthesized single stranded oligonucleotides to form duplexed barcoded adapter oligonucleotides; optionally pooling the duplexed barcoded adapter oligonucleotides; and ligating the duplexed adapter to target molecules.

Aspects of the present invention include methods of individually synthesizing hairpin oligonucleotides that contain complementary barcodes and methods of sequencing including the steps of: 1) annealing the single stranded oligos to form a hairpin oligonucleotide; 2) cleaving the non-complementary loop of the hairpin oligonucleotide adapter; and 3) ligating the adapter to the target molecule.

In one embodiment the adapters comprise a three base pair barcode. In another embodiment barcodes can contain as few as 2 or as many as 6 base pairs. To generate the pool of Y-shape duplexed adapters containing 3 base barcodes 128 oligonucleotides need to be individually synthesized or two groups of 64 adapters. The 128 oligonucleotides consist of 64 top strand and 64 complementary bottom strand oligonucleotides. When annealed to the complementary strand the 128 oligonucleotides will generate 64 Y-shape duplexed barcoded adapters. To generate the pool of Y-type adapters containing 2 base barcodes 32 oligonucleotides need to be individually synthesized. The 32 oligonucleotides consist of 16 top strand and 16 complementary bottom strand oligonucleotides. When annealed to the complementary strand the 32 oligonucleotides will generate 16 Y-shape duplexed barcoded adapters. To generate the pool of adapters containing 4 base barcodes 512 oligonucleotides need to be individually synthesized. The 512 oligonucleotides consist of 256 top strand and 256 complementary bottom stand oligonucleotides. When annealed to the complementary strand the 512 oligonucleotides will generate 256 Y-shape duplexed barcoded adapters. To generate the pool of adapters containing 5 base barcodes 2,048 oligonucleotides need to be individually synthesized. The 2,048 oligonucleotides consist of 1,024 top strand and 1,024 complementary bottom strand oligonucleotides. When annealed to the complementary strand the 2,048 oligonucleotides will generate 1,024 Y-shape duplexed barcoded adapters. To generate the pool of adapters containing 6 base barcodes 8,192 oligonucleotides need to be individually synthesized. The 8,192 oligonucleotides consist of 4,096 top strand and 4,096 complementary bottom strand oligonucleotides. When annealed to the complementary strand the 8,192 oligonucleotides will generate 4,096 Y-shape duplexed barcoded adapters.

In one embodiment the adapters comprise a three base pair barcode. In another embodiment barcodes can contain as few as 2 or as many as 6 base pairs. To generate the pool of looped adapters containing 3 base barcodes 64 oligonucleotides need to be individually synthesized. To generate the pool of looped adapters containing 2 base barcodes 16 oligonucleotides need to be individually synthesized. To generate the pool of looped adapters containing 4 base barcodes 256 oligonucleotides need to be individually synthesized. To generate the pool of looped adapters containing 5 base barcodes 1,024 oligonucleotides need to be individually synthesized. To generate the pool of looped adapters containing 6 base barcodes 4,096 oligonucleotides need to be individually synthesized.

In one embodiment adapters contain all NN, or NS and NWS barcode sequences and therefore a mixed pool of adapters could contain up to 16 different barcoded adapters. To generate a 2 base pair Y-shape duplexed barcoded adapter a total of 32 oligonucleotides need to be synthesized. When complementary pairs from the set of 32 oligonucleotides are annealed, a total of 16 Y-shape duplexed barcoded adapters are generated. However, because each adapter is individually synthesized any number of different adapters could be pooled. An NN barcode will give rise to 16 unique adapter species (8 NS and 8 NW). If the “T” base is next to the UMI (3′ end), then all 16 adapters will have a ligating “T” at the 3rd reading position on the sequence which could create monotemplate issues. To mitigate the problem for the 16 adapters that end with an A-T pair at 2nd UMI position, an additional G-C pair is added. The ligating “T” base is then at the 4^{th} position when being sequenced. Therefore, the UMI information is carried in the first 2 bases and the trailing base could be the ligating “T” (for UMIs ending with G/C) or could be “GT/CT”.

In one embodiment adapters contain all NNS and NNWS barcode sequences and therefore a mixed pool of adapters could contain up to 64 different barcoded adapters. To generate a 3 base pair Y-shape duplexed barcoded adapter a total of 128 oligonucleotides need to be synthesized. When complementary pairs from the set of 128 oligonucleotides are annealed a total of 64 Y-shape duplexed barcoded adapters are generated. However, because each adapter is individually synthesized any number of different adapters could be pooled. An NNN will give rise to 64 unique adapter species (32 NNS and 32 NNW). If the “T” base is next to the UMI (3′ end), then all 64 adapters will have a ligating “T” at the 4^{th} reading position on the sequence which could create monotemplate issues. To mitigate the problem for the 32 adapters that end with an A-T pair at the third UMI position, an additional G-C pair is added. The ligating “T” base is then at the 5^{th} position when being sequenced. Therefore, the UMI information is carried in the first 3 bases and the trailing base could be the ligating “T” (for UMIs ending with G/C) or could be “GT/CT”.

In one embodiment following oligonucleotide synthesis the individually synthesized adapters are annealed to the corresponding complementary strand to form duplexed barcoded adapters. The duplexed barcoded adapters are then pooled to form a complex library of adapters.

In one embodiment following oligonucleotide synthesis the adapters are annealed and pooled to form a complex library of adapters. In another embodiment the individually synthesized adapters are pooled and then annealed as a pool to form a complex library of adapters.

In one embodiment the individually synthesized barcoded adapters are annealed to the corresponding complementary barcoded adapter. Following annealing and hybridization the annealed barcoded adapters are pooled to form a complex mixture of barcoded adapters. This complex mixture is exposed to target nucleic acid molecules and ligase is used to tag each end of the target nucleic acids with a barcoded adapter.

In one embodiment the individually synthesized barcoded adapters are combined to form a complex mixture of barcoded adapters. This complex mixture is exposed to target nucleic acids molecules and ligase is used to tag each end of the target nucleic acids with a barcoded adapter.

In one embodiment the hairpin loop of a barcoded adapter may contain a cleavable linkage. Any convenient cleavable linkage can be employed, including nucleic acid, peptide or other chemical linkers that are sensitive to a cleaving agent. For example, a cleavable linker that includes a uracil can be cleaved by contacting with a mixture of Uracil DNA glycosylase (UDG) and the DNA glycosylase-lyase Endonuclease VIII (commercially available as the USER™ enzyme from New England Biolabs). As another example a cleavable linker includes ribonucleic acids that can be cleaved by contacting with RNase. As another example a cleavable linker includes a disulfide bond that can be cleaved by contacting with a reducing agent such as dithiothreitol.

In one embodiment the hairpin loop is cleaved but this cleavage can occur at different steps of the method. In one embodiment the cleavage occurs following ligation of the adapter to the target molecule. In another embodiment the cleavage occurs following end-repair and A-tailing (ERAT) in the ERAT buffer but prior to the ligation of the adapter to the target molecules. In another embodiment the hairpin adapter and target molecules are combined in a single tube which contains both ligase and a cleavage reagent. In yet another embodiment cleavage occurs following annealing of the single stranded adapters in adapter duplexing buffer but before ligation to the target molecule.

In one embodiment the loop of the hairpin adapter may contain an inverted repeat, a non-replicable base or sequence.

In one embodiment the loop of the hairpin adapter may remain intact, that is, no cleavage event occurs. Primers complementary to the loop region may be used to amplify the target fragment and attached barcode region. Additionally, the complementary primers may contain sample indexes and/or NGS platform specific adapter sequences.

In one embodiment the adapters permit the detection of mutations present at level below 50% are capable of being detected. Preferably mutations present at a level below 5% are capable of being detected. Preferably mutations present at a level below 1% are capable of being detected. Preferably mutations present at a level at a level 0.2% are capable of being detected. Preferably mutations present at a level of 0.1% are capable of being detected. Most preferably mutations present at the assays lower limit of detection are capable of being detected.

### BRIEF DESCRIPTION OF THE DRAWINGS

FIG. 1 illustrates a hairpin adapter containing a two base pair barcode sequence represented by the NN and complementary N′N′ sequence.

FIG. 2 illustrates adapter sequences as linear sequences from the 5′ end to the 3′ end.

FIG. 3 illustrates the initial tagging step of end repair and A-tailing. A complex mix of a two base pair barcoded adapter set is opened to prepare for ligation to the prepared target materials.

FIG. 4 illustrates the ligation of the complex mix of a two base pair barcoded adapter set and the subsequent attachment of sample indexes and NGS platform specific sequences using complementary primers.

FIG. 5 illustrates a prepared target molecule having a two base pair barcode, sample index, and NGS platform specific sequences.

FIG. 6 illustrates two versions of a barcoded hairpin adapter containing either a three base pair or four base pair barcode sequence and the use of a semi-degenerate sequence to reduce the effects of sequence monotemplates.

FIG. 7 illustrates a Bioanalyzer trace of differing oligonucleotide purification conditions, loop opening conditions, and subsequent ligation to target DNA to form an adapter-target-adapter molecule.

FIG. 8 illustrates the on-target performance of the capture in the NGS sequencing run.

FIG. 9 illustrates the sensitivity and positive predictive value of the method when used to call mutations as rare as 1% in the population in the NGS sequencing run.

FIG. 10 illustrates different oligonucleotide annealing conditions.

FIG. 11 illustrates the on-target performance of capture under varied oligonucleotide purification conditions and varied loop cleavage conditions.

FIG. 12 illustrates the sensitivity and positive predictive value of the method using varied oligonucleotide adapter purification conditions and varied looped cleavage conditions.

FIG. 13 illustrates the first 10 read cycles of a 2 base pair barcoded adapter.

FIG. 14 illustrates the annealing and hybridization strategy for 128 individually synthesized oligonucleotides (64 individually synthesized stop strand oligonucleotides and 64 individually synthesized bottom strand oligonucleotides).

FIG. 15 shows a Bioanalyzer trace comparing library yields of both the looped duplex adapters (DSv 2.1) and hybridized single stranded Y-shape adapters (DSv2.2) at varied DNA input quantities.

FIG. 16 illustrates the estimated unique, on-target molecules in each prepared library.

FIG. 17 illustrates the mean target coverage or coverage post deduplication.

FIG. 18 is a comparison of sequencing metrics and consensus analysis between the looped adapters and Y-shape adapters of the present invention and the ability of the adapters to detect ultra-low frequency variants (variants comprising 0.2%). The top charts are the sequencing metrics for the looped adapters whereas the bottom charts are the sequencing metrics for the Y-shape adapters.

FIG. 19 is a comparison of the average mean target coverage between non-barcoded adapters and barcoded adapters.

FIG. 20 illustrates the extension and fill of one strand of the duplex adapter using a polymerase and dNTPs to generate a fully duplexed barcoded adapter.

FIG. 21 illustrates the simulation of start-stop collisions under different DNA input quantities and that 2 base pair and 3 base pair barcoded adapters are sufficient to uniquely label the randomly fragmented target DNA.

FIG. 22 illustrates a 2 base barcoded Y-shape duplex adapter.

FIG. 23 illustrates the mean coverage of raw reads and mean deduplicated coverage of a target base position. The target SNP was mixed with a non-target SNP at a ratio of 0.2% (target) to 99.8% (non-target). This figure illustrates an Allele Frequency (AF) of 0.2%.

FIG. 24 illustrates the sensitivity and PPV of all variants and low frequency target SNPs (present at ≤0.2%) of the sample population using barcoded adapters.

FIG. 25 illustrates the mean deduplicated coverage of a target base position from cfDNA libraries with different inputs using barcoded adapters. The cfDNA target was mixed with a non-target sample at a ratio of 1% (target cfDNA) to 99% (non-target cfDNA).

FIG. 26 illustrates the sensitivity and PPV of target variants resulted from the cfDNA mixture with an Allele Frequency (AF) of 1%.

FIG. 27 illustrates the stability of looped duplex adapters stored at varied temperatures for three weeks.

FIG. 28 illustrates the stability of the Y-shape duplex adapters stored at varied temperatures for three weeks.

### DETAILED DESCRIPTION OF THE INVENTION

The proposed method involves the use of individually synthesized duplexed barcoded adapters in next generation sequencing methods, methods of tagging target nucleic acids, methods of individually synthesizing oligonucleotides containing barcodes, and the use of complex pools of barcoded adapters.

The proposed method involves the use of barcoded hairpin oligonucleotides in next generation sequencing methods, methods of tagging target nucleic acids, methods of individually synthesizing hairpin oligonucleotides containing complementary barcodes, and the use of complex pools of barcoded hairpin adapters.

The proposed method involves individually synthesizing oligonucleotides that contain barcode regions, next the complementary regions of the oligonucleotides are annealed to generate Y-shape barcoded adapters. The number of bases desired in the complementary barcodes determines the number of oligonucleotides that need to be synthesized. For most purposes adapters with 3 different barcodes are sufficient, although for some purposes as few as 2 or as many as 6 or more may be optimal. To generate the pool of adapters containing 3 base barcodes 128 oligonucleotides need to be synthesized. The 128 oligonucleotides consist of 64 top strand and 64 complementary bottom strand oligonucleotides. When annealed to the complementary strand the 128 oligonucleotides will generate 64 Y-shape duplexed barcoded adapters. To generate the pool of Y-type adapters containing 2 base barcodes 32 oligonucleotides need to be individually synthesized. The 32 oligonucleotides consist of 16 top strand and 16 complementary bottom strand oligonucleotides. When annealed to the complementary strand the 32 oligonucleotides will generate 16 Y-shape duplexed barcoded adapters. To generate the pool of adapters containing 4 base barcodes 512 oligonucleotides need to be individually synthesized. The 512 oligonucleotides consist of 256 top strand and 256 complementary bottom stand oligonucleotides. When annealed to the complementary strand the 512 oligonucleotides will generate 256 Y-shape duplexed barcoded adapters. To generate the pool of adapters containing 5 base barcodes 2,048 oligonucleotides need to be individually synthesized. The 2,048 oligonucleotides consist of 1,024 top strand and 1,024 complementary bottom strand oligonucleotides. When annealed to the complementary strand the 2,048 oligonucleotides will generate 1,024 Y-shape duplexed barcoded adapters. To generate the pool of adapters containing 6 base barcodes 8,192 oligonucleotides need to be individually synthesized. The 8,192 oligonucleotides consist of 4,096 top strand and 4,096 complementary bottom strand oligonucleotides. When annealed to the complementary strand the 8,192 oligonucleotides will generate 4,096 Y-shape duplexed barcoded adapters.

The proposed method involves individually synthesizing hairpin oligonucleotides that contain complementary barcodes, next the complementary regions of the hairpin oligos are annealed, the non-complementary loop of the hairpin oligo is cleaved, and the adapters containing the complementary barcodes are used as adapters for library generation. The number of bases desired in the complementary barcodes determines the number of oligonucleotides that need to be synthesized. For most purposes adapters with 3 base barcodes are sufficient, although for some purposes as few as 2 or many as 6 or more may be optimal. To generate a pool of hairpin, or looped, adapters containing a 2 base barcode 16 oligonucleotides need to be synthesized. To generate the pool of hairpin, or looped, adapters containing 3 base barcodes 64 oligonucleotides need to be synthesized. To generate a pool of hairpin, or looped, adapters containing a 4 base barcode 256 oligonucleotides need to be synthesized. To generate a pool or hairpin, or looped, adapters containing a 5 base barcode 1,024 oligonucleotides need to be synthesized. To generate a pool of hairpin, or looped, adapters containing a 6 base barcode 4,096 oligonucleotides need to be synthesized.

In certain embodiments the adapter includes one or more clamp regions, a ligation site and a region of non-complementarity such that when an adapter is ligated to both ends of a nucleic acid fragment and the adapter-ligated fragment is amplified through the region of non-complementarity the resultant nucleic acid fragments are tagged.

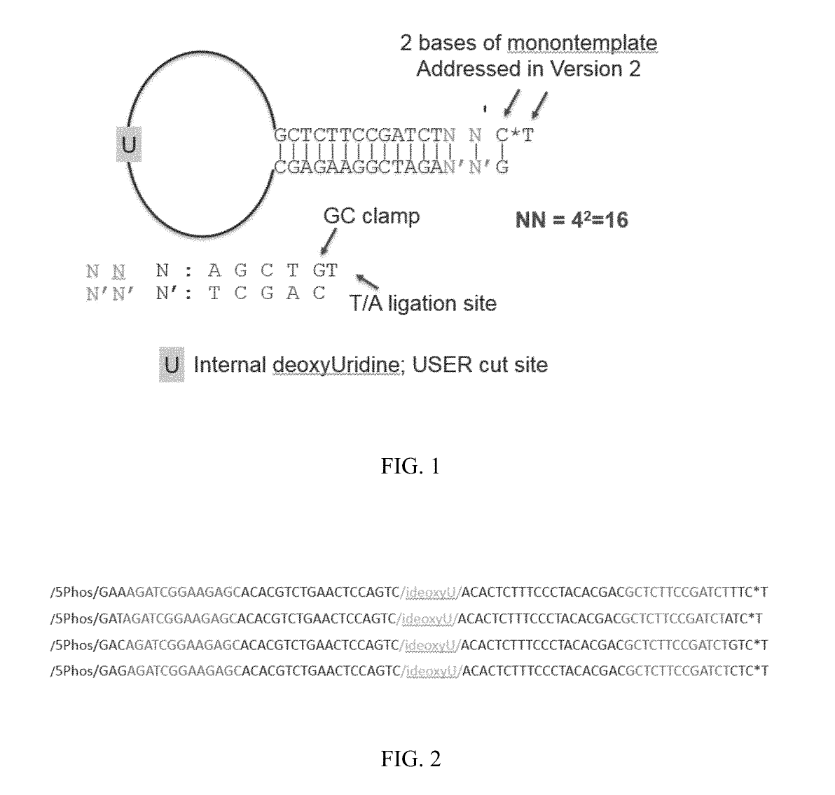

FIG. 1 shows one embodiment of the duplexed barcoded adapter containing a double stranded region and a non-complementary single stranded region. The adapter is manufactured as a single synthetic DNA sequence and following synthesis is allowed to anneal in Duplex Buffer (Integrated DNA Technologies, Inc.) to form the looped hairpin adapter. Additionally, the adapter contains a 2 base barcode (NN) region, GC clamp, and single T overhang. When using a two base barcode 16 individual adapter structures can be synthesized. Optionally the adapter can contain a cleavage region. Cleavage regions could optionally contain at least one uracil residue within the non-complementary single stranded region. Optionally the adapter may contain one or more phosphorothioate modifications.

It is noted here that the UID tag need only be a DNA sequence which uniquely identifies the sample or sample region from which the fragment so labeled originates. It is noted here that there are no constraints with regard to members of a set of tags being employed in the present invention. For example, a set of identity tags that finds use in the subject invention need not have similar thermodynamic or physical properties between them, e.g., be isothermal.

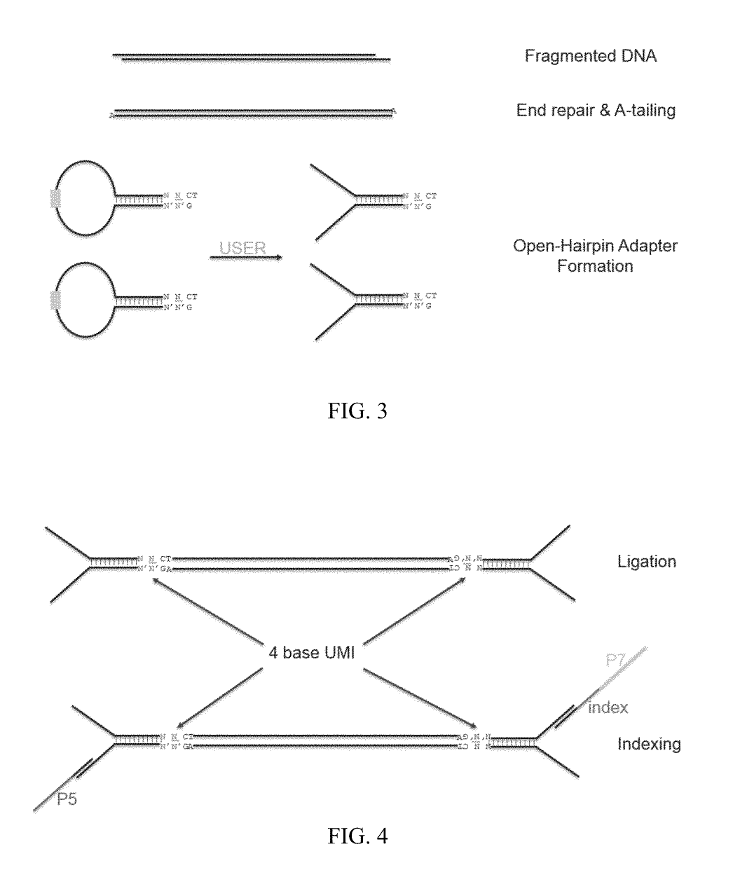

FIG. 3 shows fragmented DNA and end repaired and A-tailed target DNA. The adapters of the present invention can be ligated to both strands of the end repaired A-tailed target DNA. Furthermore FIG. 3 shows the closed and open confirmation of the barcoded adapters following cleavage of the cleavable linkage with a UDG and Endonuclease VIII mixture.

FIG. 4. shows adapter-target-adapter fragments. Sample indexes and NGS platform specific regions are added to the adapter-target-adapter fragments using primers which are complementary to the single stranded region of the adapters. Following ligation of the adapters the adapter-target-adapter fragment is denatured and sample specific primers containing sample indexes and NGS platform specific regions are allowed to anneal. Following the annealing step the target fragments are amplified by PCR generating an adapted target molecule with sample indexes and NGS platform specific regions. It should be understood that sample indexes can be added to one or both ends of the adapter-target-adapter fragment. Additionally, the use of dual matched barcoded adapters is contemplated.

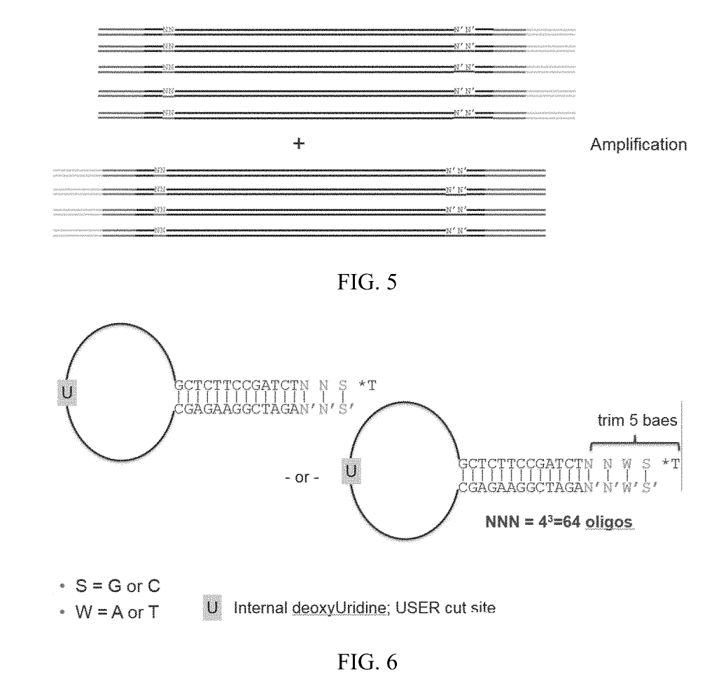

FIG. 5 shows extended adapter-target-adapter fragments (adapted target molecule) which after PCR amplification contain sample indexes, dual indexes, and NGS platform specific regions. Once extended, the tagged nucleic acid fragment can be manipulated and assayed as desired by the user. Functional regions or domains in the substantially non-complementary regions of the asymmetric adapter can facilitate such downstream analyses (e.g., sequencing, amplification, sorting based on an identity tag, etc.).

FIG. 6 illustrates an alternate embodiment of the duplexed barcoded adapter containing a double stranded region and a non-complementary single stranded region. The adapter is manufactured as a single synthetic DNA sequence and following synthesis is allowed to anneal in IDT Duplex Buffer to form the looped hairpin adapter. Additionally, adapters contain a 3 base barcode (NNS or NNW) region, GC clamp, and single T overhang. Additionally, the adapters could comprise a NNWS sequence which equates to 64 uniquely synthesized oligonucleotide adapters. S is used to represent the combination of either Guanine or Cytosine. W is used to represent the combination of either Adenine or Thymine. However, because each adapter is individually synthesized any number of different adapters could be pooled.

In another embodiment the adapters contain all NN, or NS and NWS barcode sequences and therefore a mixed pool of adapters could contain up to 16 different barcoded adapters. To generate a 2 base pair Y-shape duplexed barcoded adapter a total of 32 oligonucleotides need to be synthesized. When complementary pairs from the set of 32 oligonucleotides are annealed a total of 16 Y-shape duplexed barcoded adapters are generated. However, because each adapter is individually synthesized any number of different adapters could be pooled. An NN barcode will give rise to 16 unique adapter species (8 NS and 8 NW). If the “T” base is next to the UMI (3′ end), then all 16 adapters will have a ligating “T” at the 3rd reading position on the sequence which could create monotemplate issues. To mitigate the problem for the 16 adapters that end with an A-T pair at 2nd UMI position, an additional G-C pair is added. The ligating “T” base is then at the 4^{th} position when being sequenced. Therefore, the UMI information is carried in the first 2 bases and the trailing base could be the ligating “T” (for UMIs ending with G/C) or could be “GT/CT”. S is used to represent the combination of either Guanine or Cytosine. W is used to represent the combination of either Adenine or Thymine.

In one embodiment adapters contain all NNS and NNWS barcoded regions and therefore a mixed pool of adapters could contain up to 64 different barcoded. However, because each adapter is individually made any number of different adapters could be pooled. A NNN will give rise to 64 unique adapter species (32 NNS and 32 NNW). If the “T” base is next to the UMI (3′ end), then all 64 adapters will have this ligating “T” at the 4^{th} reading position on the sequence which could create monotemplate issues. To mitigate the problem for the 32 adapters that end with an A-T pair at the third UMI position, an additional G-C pair is added. The ligating “T” base is then at the 5^{th} position when being sequenced. Therefore, the UMI information is carried in the first 3 bases and the trailing base could be the ligating “T” (for UMIs ending with G/C) or could be “GT/CT”. S is used to represent the combination of either Guanine or Cytosine. W is used to represent the combination of either Adenine or Thymine

When using a three base barcode for looped adapters 64 individual oligonucleotide adapters are synthesized. Optionally the adapter can contain a cleavage region. Cleavage regions could optionally contain at least one uracil within the non-complementary single stranded region.

In one embodiment a semi-degenerate barcode sequence is utilized. This semi-degenerate sequence prevents monotemplate sequences that potentially affect the call efficiency. Monotemplates occur where target fragments have exactly the same sequence. By using a semi-degenerate barcode not all base reads will be identical. For example, if the nucleotide code S (representing a mix of guanine and cytosine) is used then the barcoded adapters would contain a mix of guanine and cytosine at the base. This mixed base sequence helps to ensure sufficient sequence diversity to enable accurate read calling and to reduce errors in call rates.

In one embodiment the adapters comprise a three base pair barcode. In another embodiment barcodes can contain as few as 2 or as many as 6 base pairs. To generate the pool of Y-shape duplexed adapters containing 3 base barcodes 128 oligonucleotides need to be individually synthesized or two groups of 64 adapters. The 128 oligonucleotides consist of 64 top strand and 64 complementary bottom strand oligonucleotides. When annealed to the complementary strand the 128 oligonucleotides will generate 64 Y-shape duplexed barcoded adapters. To generate the pool of Y-type adapters containing 2 base barcodes 32 oligonucleotides need to be individually synthesized. The 32 oligonucleotides consist of 16 top strand and 16 complementary bottom strand oligonucleotides. When annealed to the complementary strand the 32 oligonucleotides will generate 16 Y-shape duplexed barcoded adapters. To generate the pool of adapters containing 4 base barcodes 512 oligonucleotides need to be individually synthesized. The 512 oligonucleotides consist of 256 top strand and 256 complementary bottom stand oligonucleotides. When annealed to the complementary strand the 512 oligonucleotides will generate 256 Y-shape duplexed barcoded adapters. To generate the pool of adapters containing 5 base barcodes 2,048 oligonucleotides need to be individually synthesized. The 2,048 oligonucleotides consist of 1,024 top strand and 1,024 complementary bottom strand oligonucleotides. When annealed to the complementary strand the 2,048 oligonucleotides will generate 1,024 Y-shape duplexed barcoded adapters. To generate the pool of adapters containing 6 base barcodes 8,192 oligonucleotides need to be individually synthesized. The 8,192 oligonucleotides consist of 4,096 top strand and 4,096 complementary bottom strand oligonucleotides. When annealed to the complementary strand the 8,192 oligonucleotides will generate 4,096 Y-shape duplexed barcoded adapters. It should be understood that a pool can comprise any number of duplex barcoded adapters. For example, although a 2 base barcode adapter could theoretically generate 16 unique barcoded adapters not all 16 unique barcodes need to be pooled.

In one embodiment looped adapters comprise a three base pair barcode. In another embodiment looped adapter barcodes can contain as few as 2 base pairs or as many as 6 base pairs. To generate the pool of looped adapters containing 3 base barcodes 64 oligonucleotides need to be synthesized. To generate the pool of looped adapters containing 2 base barcodes 16 oligonucleotides need to be individually synthesized. To generate the pool of looped adapters containing 4 base barcodes 256 oligonucleotides need to be individually synthesized. To generate the pool of looped adapters containing 5 base barcodes 1024 oligonucleotides need to be individually synthesized. To generate the pool of looped adapters containing 6 base barcodes 4096 oligonucleotides need to be individually synthesized. It should be understood that a pool can comprise any number of individually synthesized adapters. For example, although a 2 base barcode adapter could theoretically generate 16 unique barcoded adapters not all 16 unique barcodes need to be pooled.

In another embodiment the barcoded adapters are pooled to form a complex mixture of adapters. For example, in one embodiment adapters containing a 2 base pair barcode would generate up to 16 distinct Y-shape duplexed barcoded adapters. The individual adapter complementary pairs may be pre-annealed prior to pooling such that each complementary pair would form a Y-shape duplexed barcoded adapter. The individual duplexed adapters are pooled at concentrations appropriate for NGS processes. The concentrations vary but can be from 1 uM to 30 uM. The complex pool of adapters is ligated to target nucleic acids creating a mixture of adapter-target-adapter molecules. The mixture of adapter-target adapter molecules is amplified by PCR. The complex pool of adapters can be formed from 64 duplexed barcoded adapters, 256 duplexed barcoded adapters, 1,024 duplexed barcoded adapters, 4,096 duplexed barcoded adapters, or any suitable combination.

In another embodiment barcoded adapters are pooled to form a complex mixture of looped adapters. For example, in one embodiment adapters containing a 2 base pair barcode generate 16 distinct oligonucleotide adapters. These individual adapters may be pre-annealed prior to pooling such that each adapter would form a hairpin, or looped, adapter. The individual hairpin adapters are pooled at concentrations appropriate for NGS processes to form a complex pool of looped adapters. This concentration varies but can be from 1 uM to 30 uM. In another embodiment the individually synthesized oligonucleotides can be pooled and then annealed as a pool to form a complex pool of looped adapters. The complex pool of looped adapters is ligated to target nucleic acids creating a mixture of adapter-target-adapter molecules. The mixture of adapter-target adapter molecules is amplified by PCR. The complex pool of adapters can be formed from 64 oligonucleotides (3 base barcode), 256 oligonucleotides (4 base barcode), 1,024 oligonucleotides (5 base barcode), 4,096 oligonucleotides (6 base barcode), or any suitable combination.

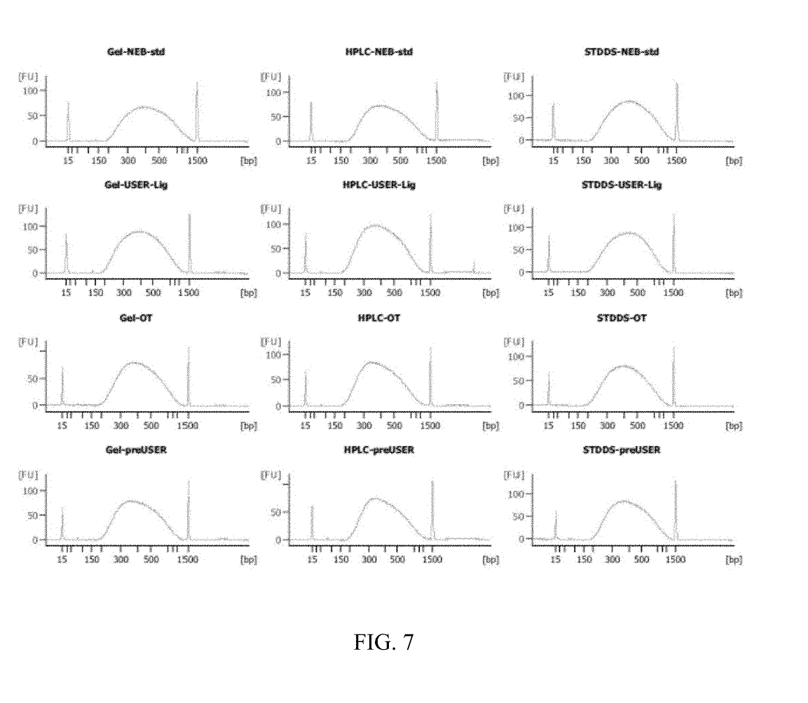

FIG. 7 shows a Bioanalyzer trace of varied oligonucleotide purification conditions, loop opening conditions, and subsequent ligation to target DNA to form an adapter-target-adapter molecule. Synthesized oligonucleotide adapters were purified using PAGE (Gel), HPLC, or standard desalting (std) procedures. The hairpin oligonucleotide adapters were cleaved under different enzymatic treatment methods which include: 1) cleavage with a UDG and Endonuclease VIII mixture after ligation of the hairpin adapters to the target molecule; 2) cleavage with a UDG and Endonuclease VIII mixture after target End-repair and A-tailing in the End-repair buffer but with the cleavage occurring prior to ligation; 3) a one tube method where adapters, prepared target nucleic acids, a UDG and Endonuclease VIII mixture, and ligase are mixed in a single tube and wherein the cleavage and ligation occurs in the same tube; and 4) a pre-cleavage of the hairpin oligonucleotide with a UDG and Endonuclease VIII mixture wherein the cleavage occurs post annealing in duplexing buffer but before ligation to the target molecule.

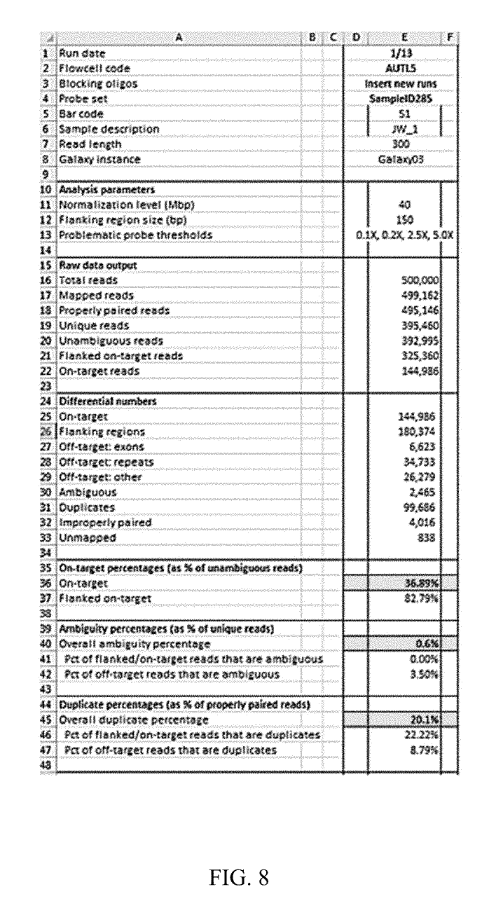

FIG. 8 shows the NGS sequencing data and shows the on-target performance of the capture. Target DNA was a mixture of NA12878 and NA24385 genomic DNA. The two genomic DNA samples were combined in a 98:2 ratio and a total of 2 ug of the mixture was used for fragmentation, end-repair and A-tailing to generate a prepared target molecule. The pooled barcoded adapters were then ligated to the prepared target molecule to form an adapter-target-adapter fragment. Prior to the adapter ligation the pre-annealed adapters were treated with a UDG and Endonuclease VIII mixture in IDT Duplex buffer to cleave the adapters. The cleaved adapters were then ligated to the fragmented target DNA mixture. The prepared library was run on an Illumina MiSeq® synthesizer and the corresponding raw sequencing data was analyzed.

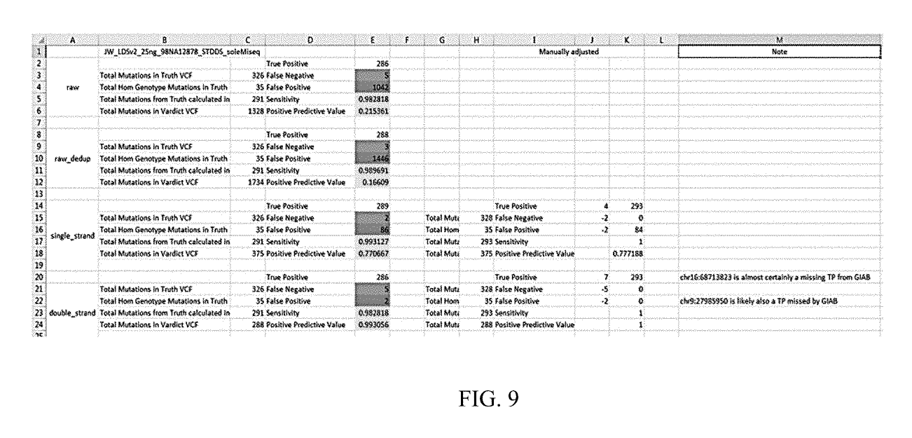

FIG. 9 shows NGS sequencing data of the pre-cleaved adapter. The data show the sensitivity and positive predictive value of the method when used to call mutations as rare as 1% in the population. Raw reads have a Sensitivity of 98.2% but a Positive Predictive Value of 21.5%. Raw deduplicated reads have a Sensitivity of 98.9% and a Positive Predictive Value of 16.6%. Single strands deduplicated reads have a Sensitivity of 99.3% and a Positive Predictive Value of 77.1%. The looped adapters deduplicated reads have a Sensitivity of 98.2% while the Positive Predictive Value is 99.3%.

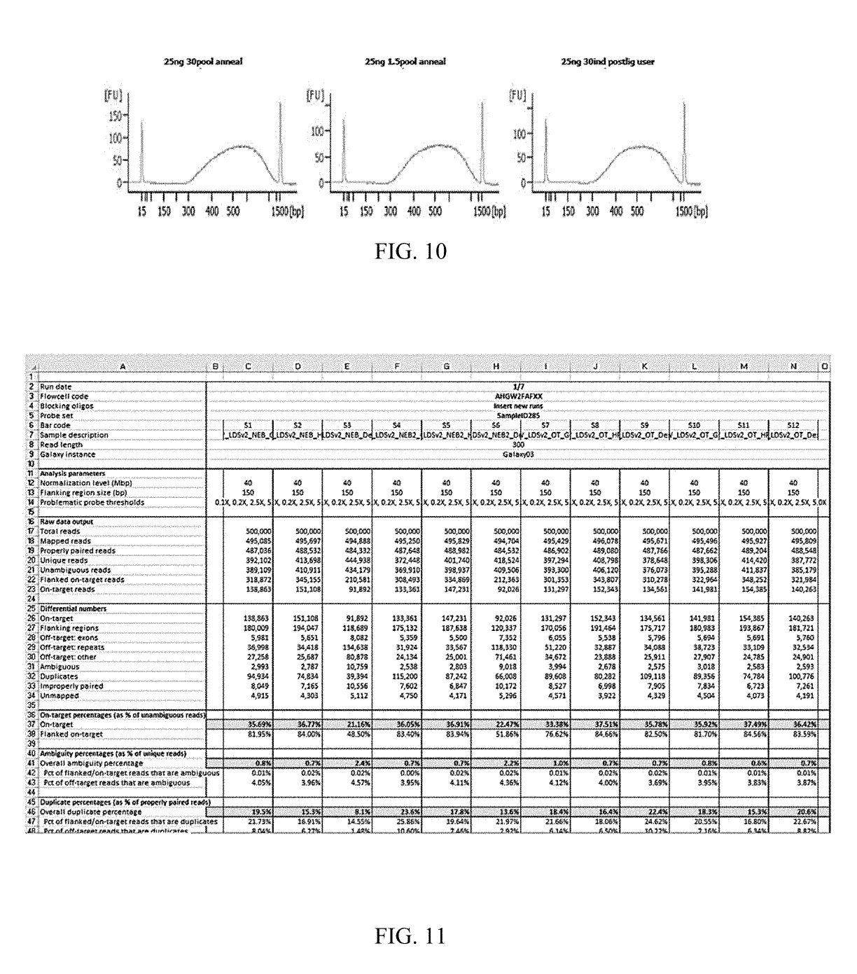

FIG. 10 illustrates different oligonucleotide annealing conditions. The first trace, 25 ng 30 pool anneal, shows 64 individually synthesized looped adapters pooled to a concentration 30 uM. The pooled looped adapters were then annealed in IDT Duplex Buffer. The pooled and annealed looped adapters were then ligated to end-repaired and A-tailed target DNA. Following ligation the adapter-target-adapter molecules were run on a Bioanalyzer.

The second trace, 25 ng 1.5 pool anneal, shows 64 individually synthesized looped adapters pooled to a concentration of 1.5 uM total. The pooled looped adapters were then annealed in IDT Duplex Buffer. The pooled and annealed looped adapters were then ligated to end-repaired and A-tailed target DNA. Following ligation the adapter-target-adapter molecules were run on a Bioanalyzer.

The third trace, 25 ng 30 ind postlig user, shows 64 individual synthesized looped adapters that are individually annealed. The individually annealed looped adapters were combined to a final concentration of 30 uM. The individually annealed and pooled looped adapters were ligated to the target molecule. Following ligation the adapter-target-adapter molecules were run on a Bioanalyzer.

FIG. 10 shows that the individually synthesized loop type adapters can be pooled and annealed as a pool or annealed individually and then pooled without loss in performance or ability to ligate efficiently to target nucleic acids.

FIG. 11 shows the on target capture percentages of the sequencing experiments. Looped oligonucleotide adapters were either purified using PAGE (Gel), HPLC, or standard desalting methods. The purified and annealed adapters were then exposed to varied cleavage and ligation conditions.

Cleavage and ligation conditions include: 1) ligating the looped adapters to the target molecule to create an adapter-target-adapter molecule which is then treated with a UDG and Endonuclease VIII mixture to cleave the adapters at the cleavable linkage (shown as S1 PAGE, S2 HPLC, and S3 Standard Desalting in FIG. 11); 2) cleavage with a UDG and Endonuclease VIII mixture in the end-repair buffer after End-repair and A-tailing of the target. However, cleavage occurs prior to the ligation of the adapters and target molecules (shown as S4 PAGE, S5 HPLC, and S6 Standard Desalting in FIG. 11); 3) a one tube method where the UDG and Endonuclease VIII mixture treatment and ligation occur in the same tube. This single tube contains pooled adapters, prepared target nucleic acids, cleavage reagents, and ligase (show in S7 PAGE, S8 HPLC, and S9 Standard Desalting in FIG. 11); and 4) a pre-cleavage treatment of the hairpin oligonucleotide in IDT duplex buffer immediately after adapter annealing reactions. The pre-cleaved adapters were then combined with target molecules and ligase to complete the ligation addition and generate an adapter-target-adapter molecule (shown as S10 PAGE, S11 HPLC, and S12 standard desalting in FIG. 11).

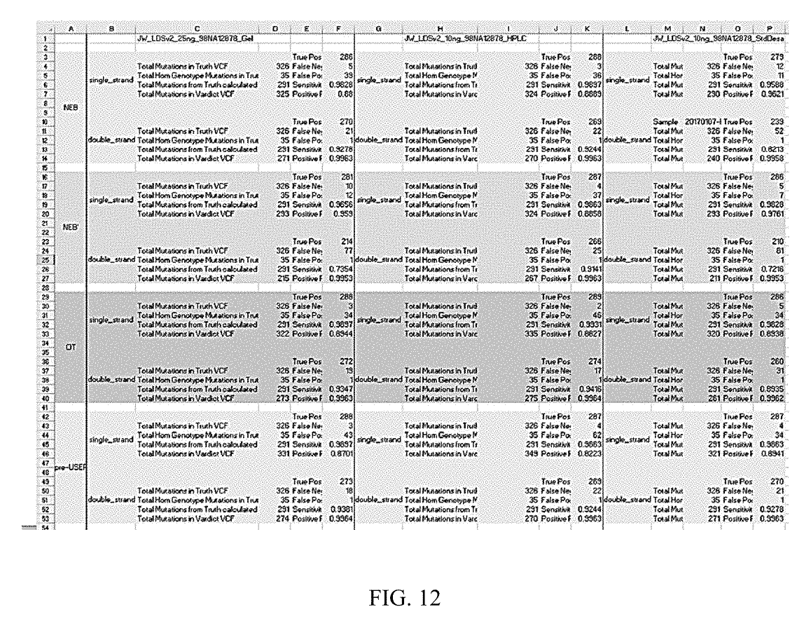

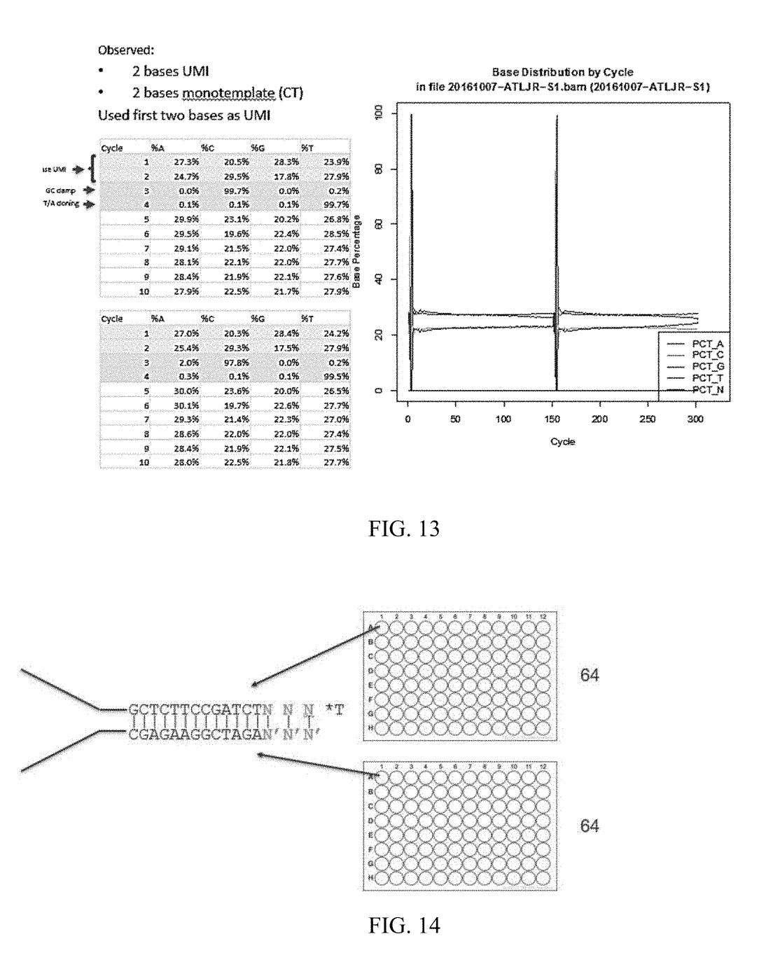

FIG. 12 shows NGS sequencing data and the sensitivity and positive predictive value. Looped oligonucleotide adapters were either purified using PAGE (Gel), HPLC, or standard desalting methods. The purified and annealed adapters were then exposed to varied cleavage and ligation conditions.

Cleavage and ligation conditions include: 1) ligating the looped adapters to the target molecule to create an adapter-target-adapter molecule. This adapter-target-adapter molecule is then treated with a UDG and Endonuclease VIII mixture to cleave the adapter at the cleavable linkage (represented by NEB); 2) Cleavage occurs after the target molecule is End-repaired and A-tailed. The cleavage occurs in the End-repair buffer but prior to ligation (represented by NEB′); 3) a one tube method where the adapters, target molecules, UDG, Endonuclease VIII, and ligase are combined into a single tube. Cleavage and ligation happen in the same tube, but due to enzyme kinetics it is expected that the cleavage happens at a faster rate (represented by OT); and 4) cleavage of the adapters in duplex buffer with a UDG and Endonuclease VIII mixture immediately after adapter annealing reactions. The pre-cleaved adapters are then combined with target molecules and ligase to complete the ligation addition and generate an adapter-target-adapter molecule (represented by pre-USER). The data show that the looped adapters generate high on target reads and provide high Sensitivity and Positive Predictive Values across a variety of adapter purification strategies and cleavage strategies.

FIG. 14 shows the annealing and hybridization strategy for a 3 base pair adapter oligonucleotide. 128 individual oligonucleotide adapters are synthesized each containing a 14 base pair common region and barcode region that is variable. This barcode region could comprise 2 base pairs, 3 base pairs, 4 base pairs, 5 base pairs, or 6 base pairs. In this figure the barcode region comprises 3 bases. It is also contemplated that a suitable barcode could comprise 2 to six bases. Following synthesis, complementary oligonucleotide pairs are combined with each other, for example well position A1 of each individually synthesized plate contains complementary sequence pairs. This is repeated for each well position, e.g., the oligonucleotide of A2 of one plate is combined with the complementary oligonucleotide of A2 of the second plate, the oligonucleotide of B1 of one plate is combined with the complementary oligonucleotide of B1 of the second plate, and the oligonucleotide of C1 of one plate is combined with the complementary oligonucleotide C2 of the second plate. This combining and annealing of the complementary pairs is repeated until the complementary pairs are combined. The complementary sequences are combined with each other in equimolar amounts and allowed to anneal and hybridize forming the desired Y-shape barcoded adapter. For example, when annealed to the respective complementary sequences the initial 128 synthesized oligonucleotides (64 top strand and 64 complementary bottom strands) will generate 64 distinct Y-shape duplexed barcoded adapters.

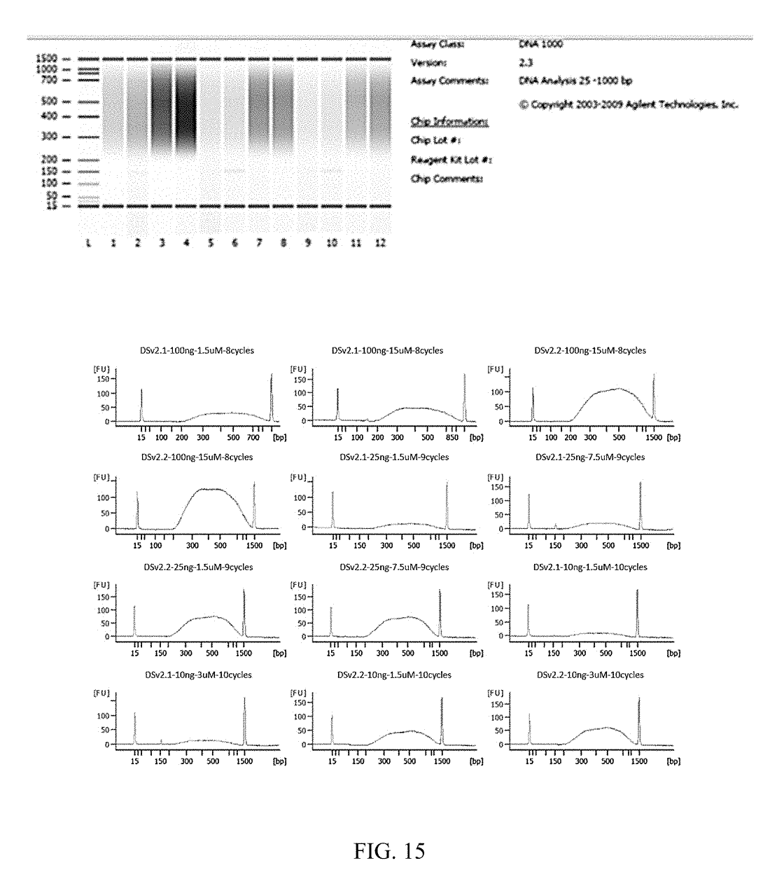

FIG. 15 shows a Bioanalyzer trace comparing library yields of both the looped duplex adapters (DSv 2.1) and hybridized single stranded Y-shape adapters (DSv2.2) at varied DNA input quantities. The figure demonstrates that both the looped adapter and Y-shape duplexed barcoded adapters are capable of generating prepared libraries suitable for next generation sequencing. Both adapter versions can effectively label target libraries at varied library concentrations, varied adapter concentrations and varied PCR cycles. The prepared libraries are suitable for next generation sequencing applications.

DSv2.1-100 ng-1.5 uM-8 cycles represents the ligation of a pool of looped adapters (v2.1) to 100 ng of sheared target DNA, with a pooled adapter input concentration of 1.5 uM. The sample was PCR amplified for 8 cycles to generate a prepared target library.

DSv2.1-100 ng-15 uM-8 cycles represents the ligation of a pool of looped adapter (v2.1) to 100 ng of sheared target DNA, with a pooled adapter input concentration of 15 uM. The sample was PCR amplified for 8 cycles to generate a prepared target library.

DSv2.2-100 ng-1.5 uM-8 cycles represents the ligation of a pool of duplexed Y-shape adapter (v2.2) to 100 ng of sheared target DNA, with a pooled adapter input concentration of 1.5 uM. The sample was PCR amplified for 8 cycles to generate a prepared target library.

DSv2.2-100 ng-1.5 uM-8 cycles represents the ligation of a pool of duplexed Y-shape adapter (v2.2) to 100 ng of sheared target DNA, with a pooled adapter input concentration of 15 uM. The sample was PCR amplified for 8 cycles to generate a prepared target library.

DSv2.1-25 ng-1.5 uM-9 cycles represents the ligation of a pool of looped adapter (v2.1) to 25 ng of sheared target DNA, with a pooled adapter input concentration of 1.5 uM. The sample was PCR amplified for 9 cycles to generate a prepared target library.

DSv2.1-25 ng-7.5 uM-9 cycles represents the ligation of a pool of looped adapter (v2.1) to 25 ng of sheared target DNA, with a pooled adapter input concentration of 7.5 uM. The sample was PCR amplified for 9 cycles to generate a prepared target library.

DSv2.2-25 ng-1.5 uM-9 cycles represents the ligation of a pool of duplexed Y-shape adapter (v2.2) to 25 ng of sheared target DNA, with a pooled adapter input concentration of 1.5 uM. The sample was PCR amplified for 9 cycles to generate a prepared target library.

DSv2.2-25 ng-7.5 uM-9 cycles represents the ligation of a pool of duplexed Y-shape adapter (v2.2) to 25 ng of sheared target DNA, with a pooled adapter input concentration of 7.5 uM. The sample was PCR amplified for 9 cycles to generate a prepared target library.

DSv2.1-10 ng-1.5 uM-10 cycles represents the ligation of a pool of looped adapter (v2.1) to 10 ng of sheared target DNA, with a pooled adapter input concentration of 1.5 uM. The sample was PCR amplified for 10 cycles to generate a prepared target library.

DSv2.1-10 ng-3 uM-10 cycles represents the ligation of a pool of looped adapter (v2.1) to 10 ng of sheared target DNA, with a pooled adapter input concentration of 3 uM. The sample was PCR amplified for 10 cycles to generate a prepared target library.

DSv2.2-10 ng-1.5 uM-10 cycles represents the ligation of pool of Y-shape adapter (v2.2) to 10 ng of sheared target DNA, with a pooled adapter input concentration of 1.5 uM. The sample was PCR amplified for 10 cycles to generate a prepared target library.

DSv2.2-10 ng-3 uM-10 cycles represents the ligation of pool of Y-shape adapter (v2.2) to 10 ng of sheared target DNA, with a pooled adapter input concentration of 3 uM. The sample was PCR amplified for 10 cycles to generate a prepared target library.

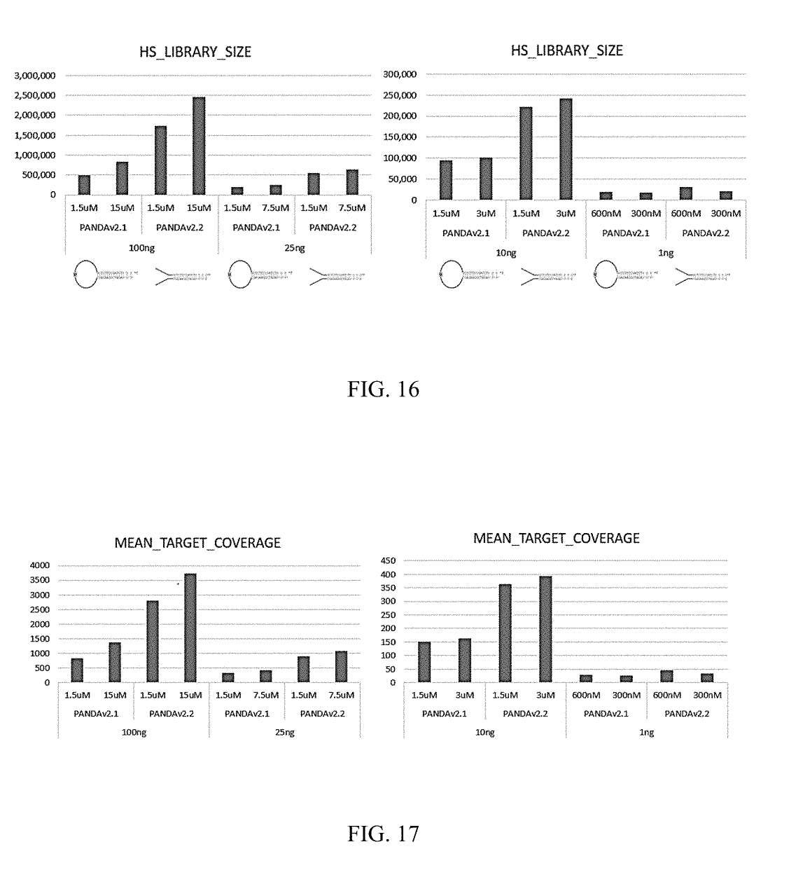

FIG. 16 illustrates the estimated unique, on-target molecules in each prepared library. Both adapter versions (looped v2.1 and Y-shape v2.2) are capable of efficiently ligating to target DNA. The adapter concentrations during ligation range from 300 nm to 15 uM. The adapter input concentrations are 15 uM, 7.5 uM, 3 uM, 1.5 uM, 600 nM, and 300 nM. Additionally, the sheared target DNA input concentrations are varied from 100 ng to 1 ng. Sheared target DNA input concentrations are 100 ng, 25 ng, 10 ng, and 1 ng. Following ligation of the adapters (either v2.1 or v2.2) the target libraries are PCR amplified and then sequenced. The adapters are capable of efficiently ligating to target DNA and generating sequencing libraries which produce high library complexity and high molecular complexity.

FIG. 17 illustrates the mean target coverage of sequencing reads post deduplication. Both adapter versions (looped v2.1 and Y-shape v2.2) are capable of efficiently ligating to target DNA. The adapter concentrations during ligation range from 300 nm to 15 uM. The adapter input concentrations are 15 uM, 7.5 uM, 3 uM, 1.5 uM, 600 nM, and 300 nM. Additionally, the sheared target DNA input concentrations are varied from 100 ng to 1 ng. Sheared target DNA input concentrations are 100 ng, 25 ng, 10 ng, and 1 ng. Following ligation of the adapters (either v2.1 or v2.2) the target libraries are PCR amplified and then sequenced. The adapters are capable of efficiently ligating to target DNA and generating sequencing libraries which produce high library complexity and high molecular complexity post deduplication. This high library complexity and high molecular complexity will provide a high mean of deduplicated target coverage.

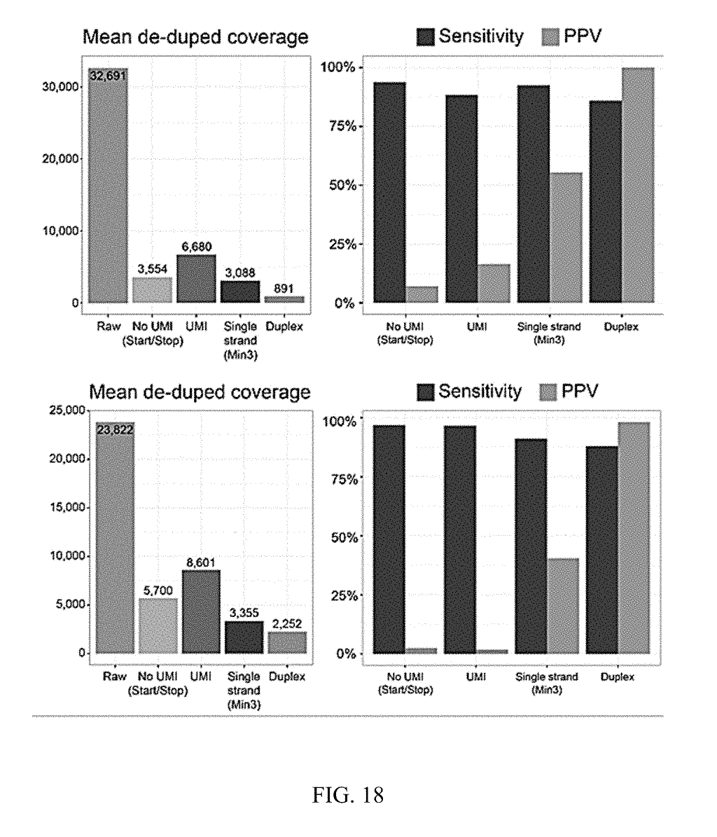

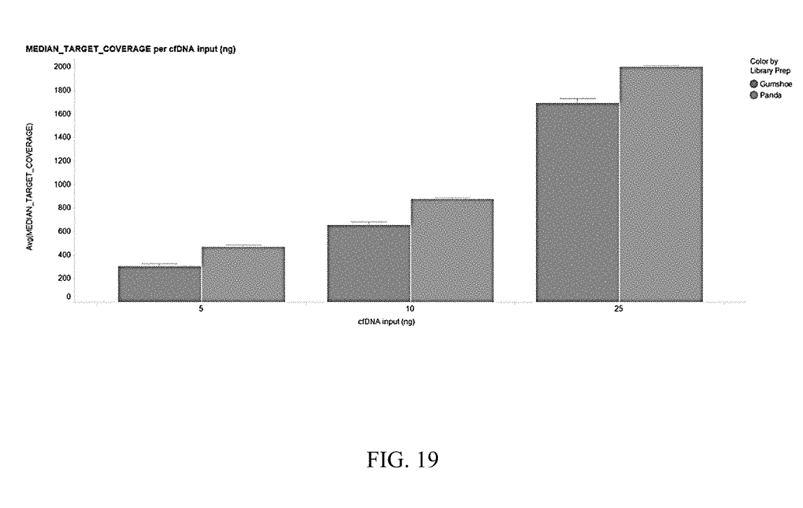

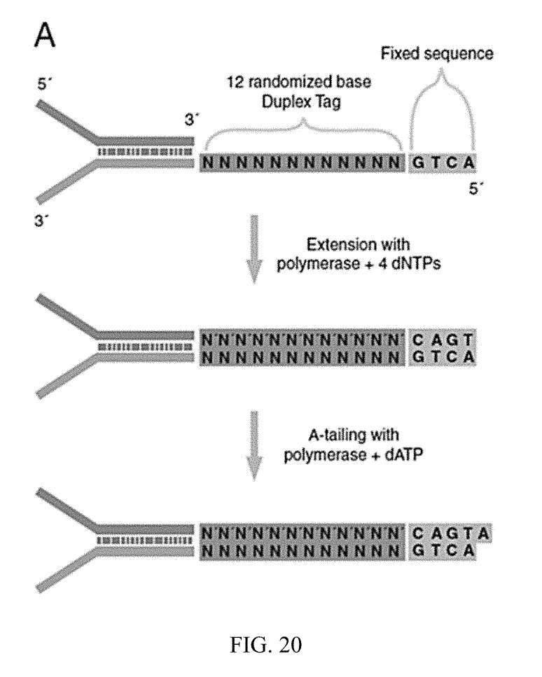

FIG. 18 is a comparison of sequencing metrics and consensus analysis between the looped adapters (DSv2.1) and Y-shape adapters (DSv2.2) of the present invention and the ability of the adapters to detect ultra-low frequency variants (variants comprising 0.2%). The top charts are the sequencing metrics for the looped adapters whereas the bottom charts are the sequencing metrics for the Y-shape adapters.

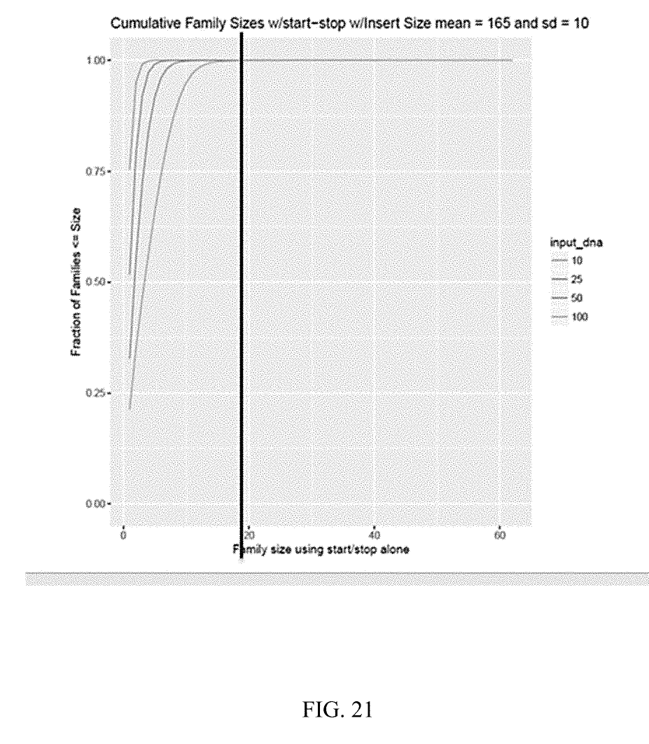

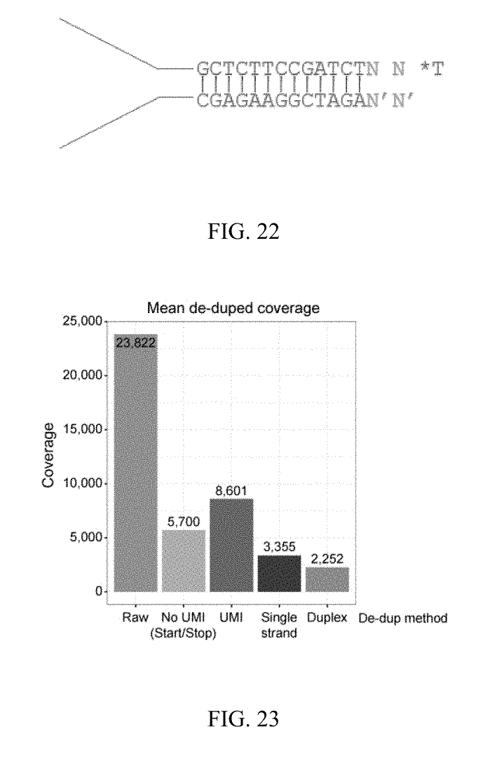

FIG. 21 illustrates the minimum number of barcoded adapters needed to uniquely label randomly sheared target DNA. The figure demonstrates that 20 unique barcoded adapters are sufficient to label 100 ng of randomly fragmented target DNA. Additionally, the figure shows that fewer unique barcodes are sufficient to uniquely label lower input quantities of randomly fragmented target DNA.

In one embodiment the duplexed adapters are capable of accurately detecting low frequency mutations. For example, DNA may be isolated from whole genomic DNA, cfDNA, FFPE DNA, circulating tumor DNA (ctDNA), or isolated from liquid biopsy. Rare mutation detection refers to detection of a sequence variant that is present at a very low frequency in a pool of wild-type (WT) background. Typically, rare variants are categorized as the variants present at or below 5% in a mixed population. Ultra-rare variants are categorized as variants present at or below 1% in a mixed population. The challenge for rare mutation, or variant, detection is the accurate discrimination between two highly similar sequences, one of which is significantly more abundant than the other.

Mutations present at level below 50% are capable of being detected. Preferably mutations present at a level below 5% are capable of being detected. Preferably mutations present at a level below 1% are capable of being detected. Preferably mutations present at a level at a level 0.2% are capable of being detected. Preferably mutations present at a level of 0.1% are capable of being detected. Most preferably mutations present at the assays lower limit of detection are capable of being detected.

FIG. 23 illustrates the mean raw and deduplicated coverages after different deduplication methods for barcoded duplex adapters. Sample NA24385 was mixed with Sample NA12878 at a ratio of 0.2% to 99.8%. The adapters are capable of efficiently ligating to target DNA and generating sequencing libraries which produce high library complexity and high molecular complexity post deduplication. This high library complexity and high molecular complexity will provide a high mean of deduplicated target coverage permitting detection of ultra-rare mutants present in the target material.

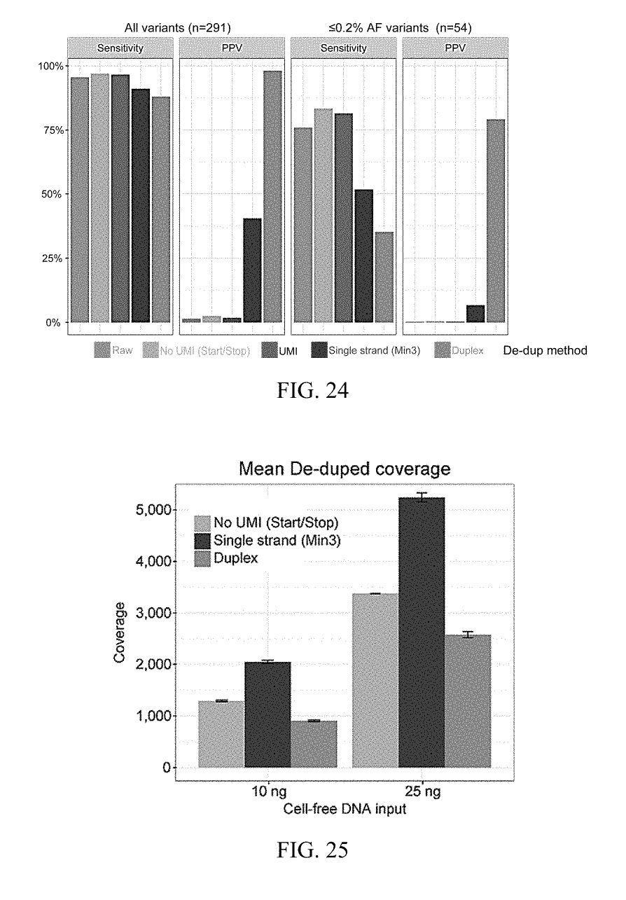

FIG. 24 illustrates the sensitivity and PPV of all variants and low frequency target SNPs (present at ≤0.2%) of the sample population using barcoded adapters. The barcoded adapters permit highly accurate variant detection for mutants present in the target material.

FIG. 25 illustrates the mean raw and deduplicated coverages after different deduplication methods for the barcoded duplex adapters. cfDNA samples were mixed at a ratio of 0.2% (cfDNA1) to 99.8% (cfDNA2). The adapters are capable of efficiently ligating to target DNA and generating sequencing libraries which produce high library complexity and high molecular complexity post deduplication. This high library complexity and high molecular complexity will provide a high mean of deduplicated target coverage permitting detection of ultra-rare mutants present in the target cfDNA material.

In one embodiment the cleavable linker includes ribonucleic acids that can be cleaved by contacting with a cleavage agent such as RNase. As another example a cleavable linker includes a disulfide bond that can be cleaved by contacting with a reducing agent such as dithiothreitol.

In another embodiment, the looped barcoded adapter is ligated to the target molecules but is not cleaved. The adapter-target-adapter molecule is amplified using at least two primers that are complementary to nucleic acid sequences within the loop. These primers may further contain sample indexes and NGS platform specific sequences.

In another embodiment, following ligation of the adapters to the target nucleic acid additional sequences may be attached to the adapter-target-adapter molecule. These additional sequences can be added enzymatically, by ligation for example, or attached through annealing of tailed complementary primers and PCR. Additional sequences may optionally include sample indexes and NGS platform specific sequences.

The method of generating error corrected sequences includes tagging each fragment of a double stranded target nucleic acid, for example dsDNA. By tagging each fragment of the dsDNA separately the sequence information of each strand is preserved. Each piece of dsDNA can produce two clonally amplified clusters of reads, each cluster originating from one strand of the original dsDNA.

In the data analysis the reliability of the reads is increased by combining the multiple reads generated by clonal amplification into a single strand consensus sequence. This single strand consensus is created from all of the PCR duplicates that arise from an individual molecule of single-stranded DNA. In the next step of the analysis the consensus sequences obtained independently from the two complementary strands present in the original DNA fragment are compared to generate a duplex consensus sequence. Because the reads from the two strands can be made independent of their errors, the method reduces the error rate by several orders of magnitude.

The following examples further illustrate the invention but, of course, should not be construed as in any way limiting its scope.

### Example 1

Generation of Hairpin Barcoded Adapters and their Use in Sequencing

This example demonstrates varied barcoded adapter hairpin purification strategies and subsequent enzymatic treatment steps.

Intra-Molecular Duplexing of UMI-Containing Oligonucleotides

64 individually synthesized single stranded oligos were resuspended in IDT Duplex Buffer at 30 μM. They were pooled with equal volume and heated to 95 C for 2 minutes. Subsequently, they were allowed to cool to room temperature and stored at −20 C freezer.

Adapter Preparation

Pooled and annealed oligos were mixed with USER enzyme (New England Biolabs) at a 5:1 V:V ratio. The mixture was incubated at 37 C water bath for 15 minutes before being stored at −20 C.

Material Preparation

Approximately 2 μg of DNA (a mixture of 98% NA12878 and 2% NA24385 genomes, both from Coriell Institute for Medical Research) was diluted in 130 μL IDTE buffer. The material was subjected to Covaris Ultrasonicator to be sheared to an average of 300 bp (10% Duty Factor, 200 Cycles per Burst, 80 seconds of treatment time) at 7 C. The sheared DNA was subsequently diluted to 15 ng per μL for next steps.

Library Construction

Libraries were prepared with NEBNext UltraII Kit (New England Biolabs, NEB) using the adapters described above. Fragmented DNA was end-repaired and adenylated at 3′ ends, followed by ligation of aforementioned adapters. The resulting DNA molecules were subjected to 0.9×SPRI clean-up and PCR-amplification using NEB's Q5 polymerase using primers that contain a sample index. PCR products were purified by a 0.9×SPRI clear-up step, which gave rise to the final whole genome libraries. Library mass was measured by Qubit (Thermo Fisher) Broad Range assay and 500 ng was used for hybridization capture with a custom IDT xGen panel, SampleID285, of 801 probes. The DNA library and capture panel were incubated overnight at 65° C., followed by binding to DYNABEADS M 450 (Thermo Fisher) beads. The beads then underwent 3 rounds of heated washes at 65° C. with IDT Wash Buffer 1 and Stringent Wash Buffer, and 3 rounds of IDT Wash Buffer 1-3. The resulting materials were subjected to a PCR amplification with primers specific to Illumina P5 and P7 sequences using KAPA HiFi Polymerase. The amplified materials were subjected to a 1.5×SPRI clean-up, which formed the final libraries for sequencing.

Analyses

Samples were sequenced in Pair-End mode (2*151) on Illumina's MiSeq or NextSeq.

Sequencing-Related Metrics

Raw base call files (.bcl files) were de-multiplexed by IDT's internal bioinformatics pipeline to generate fastq files for each read for each sample. Fastq files were aligned to the human genome (GRCh37) using BWA Mem aligner to generate sequence alignment/mapping files (.sam files), which were then utilized to produce assessment metrics using Picard tools suite.

Duplex-Sequencing Metrics

BCL files were de-multiplexed in a UMI-aware way. To be more specific, due to the defined structure of the adapters used in library preparation, the first three bases of each read correspond to the 3 UMI bases. The base calls for these 3 bases were recorded into a tag associated with the read from which the bases were from. Because of the defined structure of the adapters, the next 2 bases following the UMI bases were trimmed because they only served the purpose of providing the ligation site and were not part of UMI or genomic DNA.

After the first 5 bases were handled (3 bases of UMI and 2 trimmed bases) to form proper tags or be trimmed, the sequences were subjected to BWA MEM alignment. Then aligned reads were grouped by their UMI tag (fgbio tools suite by Fulcrum Genomics) and a consensus read was built based on all the reads with the same UMI tag fgbio). Single-stranded consensus reads were subsequently used to build, based on the complementarity of their UMI tags, double-stranded consensus reads. Variant calling is performed on single- and double-stranded consensus called reads using AstraZeneca's Vardict variant caller.

To Assess Variant Calling

Based on the documentation of Genome In A Bottle consortium, defined variants in the genomes of NA12878 and NA24385 that fall within the probe regions of IDT's xGen Lockdown SampleID285 panel are used. As the mixture of genomes is pre-defined, the frequency of each variant that is included is also calculated (For example, in a 98% NA12878 and 2% NA24385 mixture, the expected frequency of a heterozygous variant in NA24385 is 1%.). This served as the “ground truth” of the variant calling and the actual variant calls were compared against this “ground truth”. Sensitivity is calculated by diving the number of true positive variants found over the total number of expected positive (true positives/(true positives+false negatives)). Positive predictive value (PPV) is defined as the ration between the number of true positives and the number of all the positive calls (true positives/(true positives+false negatives)). Notably, homozygous mutations that exist in both NA12878 and NA24385 are not included in sensitivity and PPV.

### Example 2

The following example demonstrates varied oligonucleotide purification, loop cleavage and ligation strategies and the effects of the differential purification and cleavage strategies on on-target capture, sensitivity, and positive predictive values.

Target nucleic acid was prepared NEBNext UltraII Kit (New England Biolabs, NEB).

Barcode S1 of FIG. 11 shows PAGE purified oligonucleotide adapters, barcode S2 of FIG. 11 shows HPLC purified oligonucleotide adapters, and barcode S3 show standard desalted purified oligonucleotide adapters. Barcodes S1, S2, and S3 all underwent the same enzymatic ligation and cleavage steps. First purified and pooled annealed adapters were ligated to the end-repaired A-tailed target to create an adapter-target-adapter molecule. The adapter-target-adapter was then treated with a UDG and Endonuclease VIII mixture to cleave the adapters at the cleavable linkage.

Barcode S4 of FIG. 11 shows PAGE purified oligonucleotide adapters, barcode S5 of FIG. 11 shows HPLC purified oligonucleotide adapters, and barcode S6 show standard desalted purified oligonucleotide adapters. Pooled annealed S1, S2, and S3 purified adapters were cleaved with a UDG and Endonuclease VIII mixture after the target molecule was end-repaired and A-tailed. This cleavage occurred in the end-repair buffer. Following cleavage ligase was added and the cleaved adapters were ligated to the prepared target molecules.

Barcode S7 of FIG. 11 shows PAGE purified oligonucleotide adapters, barcode S8 of FIG. 11 shows HPLC purified oligonucleotide adapters, and barcode S9 show standard desalted purified oligonucleotide adapters. Pooled annealed S1, S2 and S3 purified adapters where added to the end-repaired and A-tailed target molecules. Ligase, UDG, and Endonuclease VIII were added to the adapter target mix and both enzymatic steps (cleavage and ligation) occurred in the same tube.

Barcodes S10 of FIG. 11 shows Page purified oligonucleotide adapters, barcode S11 of FIG. 11 shows HPLC purified oligonucleotide adapters, and barcode S12 shows standard desalted purified oligonucleotides adapters. Pooled S1, S2, and S3 purified adapters were annealed in IDT Duplex Buffer. The pre-annealed oligonucleotides adapters were cleaved with a UDG and Endonuclease VIII mixture. Following cleavage the ligase and prepared target molecules were added and the cleaved adapters were ligated to the prepared target molecules.

### Example 3

Generation of Y-Shape Barcoded Adapters and their Use in Sequencing

Inter-Molecular Annealing and Duplexing of Individually Synthesized Single Stranded UMI-Containing Oligonucleotides

128 individually synthesized single stranded oligonucleotides were suspended in IDT Duplex Buffer at 30 uM. The 128 individually synthesized single stranded oligonucleotides consist of 64 top strand oligonucleotides and 64 complementary bottom strand oligonucleotides. The complementary oligonucleotide pairs were pooled at equal volumes and heated to 95° C. for 2 minutes. Subsequently, the combined pairs were allowed to cool to room temperature and stored at −20° C. FIG. 14 demonstrates the pairing and hybridization strategy for the 128 individually synthesized single stranded oligonucleotides.

Material Preparation

Approximately 2 μg of DNA (a mixture of 98% NA12878 and 2% NA24385 genomes, both from Coriell Institute for Medical Research) was diluted in 130 μL IDTE buffer. The material was subjected to Covaris Ultrasonicator to be sheared to an average of 300 bp (10% Duty Factor, 200 Cycles per Burst, 80 seconds of treatment time) at 7 C. The sheared DNA was subsequently diluted to 15 ng per μL for next steps.

Library Construction

Libraries were prepared with KAPA Hyper Prep Kit (KAPA Biosystems) using the adapters described above. Fragmented DNA was end-repaired and adenylated at 3′ ends, followed by ligation of aforementioned adapters. The resulting DNA molecules were subjected to 0.8×SPRI clean-up and PCR-amplification using KAPA's HiFi polymerase using primers that contain a sample index. PCR products were purified by a 1×SPRI clear-up step, which gave rise to the final whole genome libraries. Library mass was measured by Qubit (Thermo Fisher) Broad Range assay and 500 ng was used for hybridization capture with a custom IDT xGen panel, SampleID285, of 801 probes. The DNA library and capture panel were incubated overnight at 65° C., followed by binding to DYNABEADS M 450 (Thermo Fisher) beads. The beads then underwent 3 rounds of heated washes at 65° C. with IDT Wash Buffer 1 and Stringent Wash Buffer, and 3 rounds of IDT Wash Buffer 1-3. The resulting materials were subjected to a PCR amplification with primers specific to Illumina P5 and P7 sequences using KAPA HiFi Polymerase. The amplified materials were subjected to a 1.5×SPRI clean-up, which formed the final libraries for sequencing

Analyses

Samples were sequenced in Pair-End mode (2*151) on Illumina's MiSeq or NextSeq.

Sequencing-Related Metrics

Raw base call files (.bcl files) were de-multiplexed by DT's internal bioinformatics pipeline to generate fastq files for each read for each sample. Fastq files were aligned to the human genome (GRCh37) using BWA Mem aligner to generate sequence alignment/mapping files (.sam files), which were then utilized to produce assessment metrics using Picard tools suite.

Duplex-Sequencing Metrics

BCL files were de-multiplexed in a UMI-aware way. To be more specific, due to the defined structure of the adapters used in library preparation, the first three bases of each read correspond to the 3 UMI bases. The base calls for these 3 bases were recorded into a tag associated with the read from which the bases were from. Because of the defined structure of the adapters, the next 2 bases following the UMI bases were trimmed because they only served the purpose of providing the ligation site and were not part of UMI or genomic DNA.

After the first 5 bases were handled (3 bases of UMI and 2 trimmed bases) to form proper tags or be trimmed, the sequences were subjected to BWA MEM alignment. Then aligned reads were grouped by their UMI tag (fgbio tools suite by Fulcrum Genomics) and a consensus read was built based on all the reads with the same UMI tag fgbio). Single-stranded consensus reads were subsequently used to build, based on the complementarity of their UMI tags, double-stranded consensus reads. Variant calling is performed on single- and double-stranded consensus called reads using AstraZeneca's Vardict variant caller.

To Assess Variant Calling

Based on the documentation of Genome In A Bottle consortium, defined variants in the genomes of NA12878 and NA24385 that fall within the probe regions of DT's xGen Lockdown SampleID285 panel are used. As the mixture of genomes is pre-defined, the frequency of each variant that is included is also calculated (For example, in a 98% NA12878 and 2% NA24385 mixture, the expected frequency of a heterozygous variant in NA24385 is 1%). This served as the “ground truth” of the variant calling and the actual variant calls were compared against this “ground truth”. Sensitivity is calculated by diving the number of true positive variants found over the total number of expected positive (true positives/(true positives+false negatives)). Positive predictive value (PPV) is defined as the ration between the number of true positives and the number of all the positive calls (true positives/(true positives+false negatives)). Notably, homozygous mutations that exist in both NA12878 and NA24385 are not included in sensitivity and PPV.

### Example 4

This example demonstrates the ability of the barcoded adapters to accurately detect low frequency or rare mutants, present in the sample DNA.

Material Preparation

Approximately 2 μg of DNA (a mixture of 99.8% NA12878 and 0.2% NA24385 genomes, both from Coriell Institute for Medical Research) was diluted in 130 μL IDTE buffer. The material was subjected to Covaris Ultrasonicator to be sheared to an average of 300 bp (10% Duty Factor, 200 Cycles per Burst, 80 seconds of treatment time) at 7 C. The sheared DNA was subsequently diluted to 15 ng per μL for next steps.

Library Construction

Libraries were prepared with KAPA Hyper Kit. 500 ng of library was put into target enrichment using MT SampleID285 custom panel as previously described.

Analyses

Samples were sequenced in air-End mode (2*151) on Illumina's MiSeq or NextSeq.

Sequencing-Related Metrics

Raw base call files (.bcl files) were de-multiplexed by IDT's internal bioinformatics pipeline to generate fastq files for each read for each sample. Fastq files were aligned to the human genome (GRCh37) using BWA Mem aligner to generate sequence alignment/mapping files (.sam files), which were then utilized to produce assessment metrics using Picard tools suite.

Duplex-Sequencing Metrics

BCL files were de-multiplexed in a UMI-aware way. To be more specific, due to the defined structure of the adapters used in library preparation, the first three bases of each read correspond to the 3 UMI bases. The base calls for these 3 bases were recorded into a tag associated with the read from which the bases were from. Because of the defined structure of the adapters, the next 2 bases following the UMI bases were trimmed because they only served the purpose of providing the ligation site and were not part of UMI or genomic DNA.

After the first 5 bases were handled (3 bases of UMI and 2 trimmed bases) to form proper tags or be trimmed, the sequences were subjected to BWA MEM alignment. Then aligned reads were grouped by their UMI tag (fgbio tools suite by Fulcrum Genomics) and a consensus read was built based on all the reads with the same UMI tag fgbio). Single-stranded consensus reads were subsequently used to build, based on the complementarity of their UMI tags, double-stranded consensus reads. Variant calling is performed on single- and double-stranded consensus called reads using AstraZeneca's Vardict variant caller.

To Assess Variant Calling

Based on the documentation of Genome In A Bottle consortium, defined variants in the genomes of NA12878 and NA24385 that fall within the probe regions of IDT's xGen Lockdown SampleID285 panel are used. As the mixture of genomes is pre-defined, the frequency of each variant that is included is also calculated (For example, in a 98% NA12878 and 2% NA24385 mixture, the expected frequency of a heterozygous variant in NA24385 is 1%.). This served as the “ground truth” of the variant calling and the actual variant calls were compared against this “ground truth”. Sensitivity is calculated by diving the number of true positive variants found over the total number of expected positive (true positives/(true positives+false negatives)). Positive predictive value (PPV) is defined as the ration between the number of true positives and the number of all the positive calls (true positives/(true positives+false negatives)). Notably, homozygous mutations that exist in both NA12878 and NA24385 are not included in sensitivity and PPV.

FIG. 23 illustrates raw or duplicate aware mean target coverages. No UMI (Start/Stop) deduplication utilizes only the position to which a fragment aligns to identify duplicates. UMI deduplication adds the tag information in addition to the genomic position in finding duplicates. Single strand (Min3) analysis collapses reads that have been grouped to the same family based on their alignment and UMIs. Duplex analysis further collapses the single strand consensus reads by finding complementary tags in a read family. The adapters are capable of efficiently ligating to target DNA and generating sequencing libraries which produce high library complexity and high molecular complexity post deduplication. This high library complexity and high molecular complexity will provide a high mean of deduplicated target coverage permitting detection of ultra-rare mutants present in the target genetic material.

FIG. 24 illustrates that sensitivity is correlated with the coverage measured with each deduplication method while the positive predictive value (PPV) was largely dictated by the degree of molecular tagging and read consensus reconstruction for low frequency variant detection.

### Example 5

This example demonstrates the ability of the barcoded adapters to accurately detect low frequency, rare mutants, and ultra-rare, present in cfDNA.

Material Preparation

Extracted cfDNA samples were purchased from Biochain. Each sample contains ˜500 ng of cfDNA material. cfDNA1 and cfDNA2 were normalized to be at 0.5 ng/uL concentration and a mixture cfDNA1 and cfDNA2 was made by mixing them at a V:V ratio.

Library Construction

Libraries were prepared with KAPA Hyper Kit. 10 ng or 25 ng of cfDNA were used as input of library and were enriched using IDT SampleID285 custom panel.

Library Sequencing and Analysis

Shallow sequencing (raw coverage 2,000×) was done using Illumina MiSeq and variants are called on the SampleID target region. The variant calls made are compared across the three samples and only those that are present in all three are considered a real mutation. The list of real mutations is used as the ground truth for evaluation of variant calling performance in the mixing experiment

FIG. 25 illustrates the mean deduplicated coverage for cfDNA target input. The cfDNA target was mixed with a non-target sample at a ratio of 1% (target cfDNA) to 99% (non-target cfDNA). No UMI (Start/Stop) deduplication utilizes only the position to which a fragment aligns to identify duplicates. UMI deduplication adds the tag information in addition to the genomic position in finding duplicates. Single strand (Min3) analysis collapses reads that have been grouped to the same family based on their alignment and UMIs. Duplex analysis further collapses the single strand consensus reads by finding complementary tags in a read family. The adapters are capable of efficiently ligating to target DNA and generating sequencing libraries which produce high library complexity and high molecular complexity post deduplication. This high library complexity and high molecular complexity will provide a high mean of deduplicated target coverage permitting detection of ultra-rare mutants present in the target cfDNA material.

### Example 6

This example demonstrates the stability of both the looped barcoded adapter and Y-shape duplex barcoded adapters.

Following annealing and duplexing of the adapters the adapters were stored at 37° C., room temperature, 4° C., and −20° C. The prepared adapters were stored for three weeks at the respective temperatures. The looped barcoded adapters (vDS2.1) were stored at either 30 uM or 1.5 uM. The Y-shape duplexed barcoded adapters (DSv2.2) were stored at 25 uM.

Following adapter storage adapter-target libraries were constructed using NEB's Ultra™ II DNA Library Prep Kit or KAPA's Hyper Prep Kit. 10 ng a sheared NA12878 was used as target DNA input for the library construction. Following library construction the prepared libraries were analyzed on a Bioanalyzer.

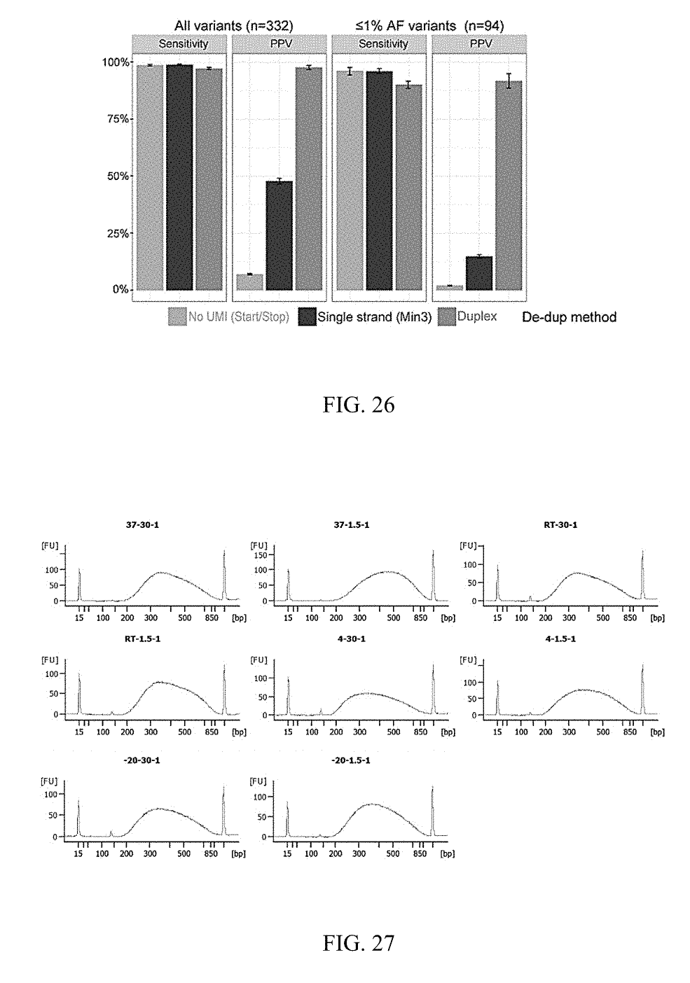

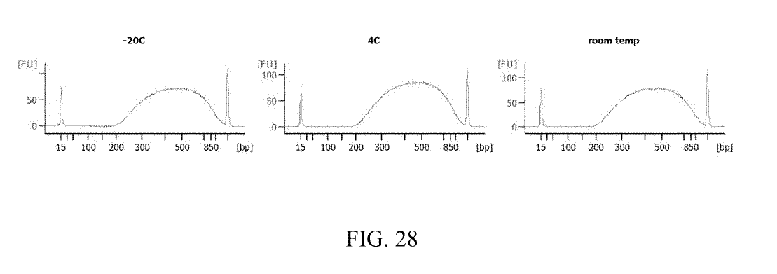

FIG. 26 demonstrates the stability of the looped barcoded (DSv2.1) adapters. The figure demonstrates that the looped barcoded adapters are stable across a range of storage temperatures and concentrations.

In the first Bioanalyzer trace of FIG. 26, 37-30-1, shows the prepared library using the looped barcoded adapters stored at 37° C. for 3 weeks at a storage concentration of 30 uM.

The second Bioanalyzer trace of FIG. 26, 37-1.5-1, shows the prepared library using the looped barcoded adapters stored at 37° C. for 3 weeks at a storage concentration of 1.5 uM.

The third Bioanalyzer trace of FIG. 26, RT-30-1, shows the prepared library using the looped barcoded adapters stored at Room-temperature for 3 weeks at a storage concentration of 30 uM.

The fourth Bioanalyzer trace of FIG. 26, RT-1.5-1, shows the prepared library using the looped barcoded adapters stored at room temperature for 3 weeks at a storage concentration of 1.5 uM.

The fifth Bioanalyzer trace of FIG. 26, 4-30-1, shows the prepared library using the looped barcoded adapters stored at 4° C. for 3 weeks at a storage concentration of 30 uM.

The sixth Bioanalyzer trace of FIG. 26, 4-1.5-1, shows the prepared library using the looped barcoded adapters stored at 4° C. for 3 weeks at a storage concentration of 15 uM.

The seventh Bioanalyzer trace of FIG. 26, −20-30-1, shows the prepared library using the looped barcoded adapters stored at −20° C. for 3 weeks at a storage concentration of 30 uM.

The eighth Bioanalyzer trace of FIG. 26, −20-1.5-1, shows the prepared library using the looped barcoded adapters stored at −20° C. for 3 weeks at a storage concentration of 1.5 uM.

FIG. 27 demonstrates the stability of the barcoded adapters (DSv2.2). The figure demonstrates that the barcoded adapters are stable across a range of storage temperatures.

The first Bioanalyzer trace of FIG. 27, −20 C, shows the prepared library using the duplex barcoded adapters stored at −20° C. for three weeks at a storage concentration of 25 uM.

The second Bioanalyzer trace of FIG. 27, 4 C, shows the prepared library using the duplex barcoded adapters stored at 4° C. for three weeks at a storage concentration of 25 uM.

The third Bioanalyzer trace of FIG. 27, room temperature, shows the prepared library using the duplex barcoded adapters stored at room temperature for three weeks at a storage concentration of 25 uM.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention

Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Deduplication” refers to the removal of reads that are determined to be duplicates from the analysis. Reads are determined to be duplicates if they share the same start stop sequences and/or UMI sequences. One purpose of deduplication is to create a consensus sequence whereby those duplicates which contain errors are removed from the analysis. Another purpose of deduplication is to estimate the complexity of the library. A library's complexity or size refers to the number of individual sequence reads that represent unique, original fragments and that map to the sequence being analyzed.

“Start stop collision” Refers to the occurrence of multiple unique fragments that contain the same start stop sites. Due to the rarity of start stop collisions, they are usually only observed when either performing ultra-deep sequencing with a very high number of reads, such as when performing rare variant detection, or when working with DNA samples that have a small size distribution such as plasma DNA. As such, start stop sites by not be enough in those scenarios since one would run the risk of erroneously removing unique fragments, mistaken as duplicates, during the deduplication step. In these case, the incorporation of barcodes into the workflow can potentially rescue a lot of complexity.

“PPV”, or Positive Predictive Value, is the probability that a sequence called as unique is actually unique. PPV=true positive/(true positive+false positive). “Sensitivity” is the probability that a sequence that is unique will be called as unique. Sensitivity=true positive/(true positive+false negative).

The term “UMI”, or “Unique Molecular Identifier”, as used herein, refers to a tag, consisting of a sequence of degenerate or varying bases, which is used to label original molecules in a sheared nucleic acid sample. In theory, due to the extremely large number of different UMI sequences that can be generated, no two original fragments should have the same UMI sequence. As such, UMIs can be used to determine if two similar sequence reads are each derived from a different, original fragment or if they are simply duplicates, created during PCR amplification of the library, which were derived from the same original fragment.

UMIs are especially useful, when used in combination with start stop sites, for consensus calling of rare sequence variants. For example, if two fragments have the same start and stop site but have a different UMI sequences, what would otherwise have been considered two clones arising from the same original fragment can now be properly designated as unique molecules. As such, the use of UMIs combined with start stop often leads to a jump in the coverage number since unique fragments that would have been labeled as duplicates using start stop alone will be labelled as unique from each other due to them having different UMIs. It also helps improve the Positive Predictive Value (“PPV”) by removing false positives. There is currently a lot of demand for UMIs, as there are some rare variants that can only be found via consensus calling using UMIs.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick base pairing with a nucleotide in the other strand. A stable duplex can include Watson-Crick base pairing and/or non-Watson-Crick base pairing between the strands of the duplex (where base pairing means the forming hydrogen bonds). In certain embodiments, a non-Watson-Crick base pair includes a nucleoside analog, such as deoxyinosine, 2,6-diaminopurine, PNAs, LNA's and the like. In certain embodiments, a non-Watson-Crick base pair includes a “wobble base”, such as deoxyinosine, 8-oxo-dA, 8-oxo-dG and the like, where by “wobble base” is meant a nucleic acid base that can base pair with a first nucleotide base in a complementary nucleic acid strand but that, when employed as a template strand for nucleic acid synthesis, leads to the incorporation of a second, different nucleotide base into the synthesizing strand (wobble bases are described in further detail below). A “mismatch” in a duplex between two oligonucleotides or polynucleotides means that a pair of nucleotides in the duplex fails to undergo Watson-Crick bonding.

Adapters are polynucleotides (either single-stranded or double-stranded) containing internal sequences complementary to each other that are capable of annealing to each other to form a duplex under appropriate conditions. Single-stranded adapters have a single-stranded loop on a first end and an opposing second end ligatable to the fragments of cleaved sample DNA.

The term “reaction mixture,” as used herein, refers to a solution containing reagents necessary to carry out a given reaction. A “ligation reaction mixture”, which refers to a solution containing regents necessary to carry out a ligation reaction, typically contains donor and acceptor oligonucleotides and a ligase in a suitable buffer. An “amplification reaction mixture”, which refers to a solution containing reagents necessary to carry out an amplification reaction, typically contains oligonucleotide primers and a DNA polymerase or ligase in a suitable buffer. A reaction mixture is referred to as complete if it contains all reagents necessary to enable the reaction, and incomplete if it contains only a subset of the necessary reagents. It will be understood by one of skill in the art that reaction components are routinely stored as separate solutions, each containing a subset of total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction components which includes the duplexed barcoded adapters and looped barcoded adapters of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

TABLE 1
oligonucleotide sequences for barcoded duplexed Y-shape adapters and
looped adapters.

| SEQ ID | | | |
|---|---|---|---|
| NO: | Sequence name | Sequence | Adapter Type |
| SEQ ID | 3 bp | 5Phos/SWNNAGATCGGAAGAGCACACGTCTGAACTCCAGTC | Y-Shape 5′Strand |
| NO: 1 | Monotemplate | | |
| | NNWS_5′ | | |
| SEQ ID | 3 bp | 5Phos/SNNAGATCGGAAGAGCACACGTCTGAACTCCAGTC | Y-Shape 5′Strand |
| NO: 2 | Monotemplate | | |
| | NNS_5′ | | |
| SEQ ID | 3 bp | ACACTCTTTCCCTACACGACGCTCTTCCGATCTN′N′W′S′*T | Y-Shape 3′Strand |
| NO: 3 | Monotemplate | | |
| | NNWS_3′ | | |
| SEQ ID | 3 bp | ACACTCTTTCCCTACACGACGCTCTTCCGATCTN′N′S′*T | Y-Shape 3′Strand |
| NO: 4 | Monotemplate | | |
| | NNS_3′ | | |
| SEQ ID | 2 bp | 5Phos/SWNAGATCGGAAGAGCACACGTCTGAACTCCAGTC | Y-Shape 5′Strand |
| NO: 5 | Monotemplate | | |
| | NWS_5′ | | |
| SEQ ID | 2 bp | 5Phos/SNAGATCGGAAGAGCACACGTCTGAACTCCAGTC | Y-Shape 5′Strand |
| NO: 6 | Monotemplate | | |
| | NS_5′ | | |
| SEQ ID | 2 bp | ACACTCTTTCCCTACACGACGCTCTTCCGATCTN′W′S′*T | Y-Shape 3′Strand |
| NO: 7 | Monotemplate | | |
| | NWS_3′ | | |
| SEQ ID | 2 bp | ACACTCTTTCCCTACACGACGCTCTTCCGATCTN′S′*T | Y-Shape 3′Strand |
| NO: 8 | Monotemplate | | |
| | NS_3′ | | |
| SEQ ID | 3 bp | 5Phos/SWNNAGATCGGAAGAGCACACGTCTGAACTCCAGTC/ | Looped |
| NO: 9 | Monotemplate | ideoxyU/ACACTCTTTCCCTACACGACGCTCTTCCGATCTN′N′ | |
| | NNWS | W′S′*T | |
| SEQ ID | 3 bp | /5Phos/SNNAGATCGGAAGAGCACACGTCTGAACTCCAGTC/ | Looped |
| NO: 10 | Monotemplate | ideoxyU/ACACTCTTTCCCTACACGACGCTCTTCCGATCTN′N′ | |
| | NNS | S′*T | |
| SEQ ID | duplex_3bp_1_5 | 5Phos/CAAAAGATCGGAAGAGCACACGTCTGAACTCCAGTC | Y-Shape 5′-strand |
| NO: 11 | | | |
| SEQ ID | duplex_3bp_2_5′ | 5Phos/CACCAGATCGGAAGAGCACACGTCTGAACTCCAGTC | Y-Shape 5′-strand |
| NO: 12 | | | |
| SEQ ID | duplex_3bp_3_5′ | 5Phos/CAGGAGATCGGAAGAGCACACGTCTGAACTCCAGTC | Y-Shape 5′-strand |
| NO: 13 | | | |
| SEQ ID | duplex_3bp_4_5′ | 5Phos/CATTAGATCGGAAGAGCACACGTCTGAACTCCAGTC | Y-Shape 5′-strand |
| NO: 14 | | | |
| SEQ ID | duplex_3bp_5_5′ | 5Phos/CAACAGATCGGAAGAGCACACGTCTGAACTCCAGTC | Y-Shape 5′-strand |
| NO: 15 | | | |
| SEQ ID | duplex_3bp_6_5′ | 5Phos/CACGAGATCGGAAGAGCACACGTCTGAACTCCAGTC | Y-Shape 5′-strand |
| NO: 16 | | | |
| SEQ ID | duplex_3bp_7_5′ | 5Phos/CAGTAGATCGGAAGAGCACACGTCTGAACTCCAGTC | Y-Shape 5′-strand |
| NO: 17 | | | |
| SEQ ID | duplex_3bp_8_5′ | 5Phos/CATAAGATCGGAAGAGCACACGTCTGAACTCCAGTC | Y-Shape 5′-strand |
| NO: 18 | | | |
| SEQ ID | duplex_3bp_9_5′ | 5Phos/GAAGAGATCGGAAGAGCACACGTCTGAACTCCAGTC | Y-Shape 5′-strand |
| NO: 19 | | | |
| SEQ ID | duplex_3bp_10_5′ | 5Phos/GACTAGATCGGAAGAGCACACGTCTGAACTCCAGTC | Y-Shape 5′-strand |
| NO: 20 | | | |
| SEQ ID | duplex_3bp_11_5′ | 5Phos/GAGAAGATCGGAAGAGCACACGTCTGAACTCCAGTC | Y-Shape 5′-strand |
| NO: 21 | | | |
| SEQ ID | duplex_3bp_12_5′ | 5Phos/GATCAGATCGGAAGAGCACACGTCTGAACTCCAGTC | Y-Shape 5′-strand |
| NO: 22 | | | |
| SEQ ID | duplex_3bp_13_5 | 5Phos/GAATAGATCGGAAGAGCACACGTCTGAACTCCAGTC | Y-Shape 5′-strand |
| NO: 23 | | | |
| SEQ ID | duplex_3bp_14_5′ | 5Phos/GACAAGATCGGAAGAGCACACGTCTGAACTCCAGTC | Y-Shape 5′-strand |
| NO: 24 | | | |
| SEQ ID | duplex_3bp_15_5′ | 5Phos/GAGCAGATCGGAAGAGCACACGTCTGAACTCCAGTC | Y-Shape 5′-strand |
| NO: 25 | | | |
| SEQ ID | duplex_3bp_16_5′ | 5Phos/GATGAGATCGGAAGAGCACACGTCTGAACTCCAGTC | Y-Shape 5′-strand |
| NO: 26 | | | |
| SEQ ID | duplex_3bp_17_5′ | 5Phos/CAAAGATCGGAAGAGCACACGTCTGAACTCCAGTC | Y-Shape 5′-strand |
| NO: 27 | | | |
| SEQ ID | duplex_3bp_18_5′ | 5Phos/CCCAGATCGGAAGAGCACACGTCTGAACTCCAGTC | Y-Shape 5′-strand |
| NO: 28 | | | |
| SEQ ID | duplex_3bp_19_5′ | 5Phos/CGGAGATCGGAAGAGCACACGTCTGAACTCCAGTC | Y-Shape 5′-strand |
| NO: 29 | | | |
| SEQ ID | duplex_3bp_20_5′ | 5Phos/CTTAGATCGGAAGAGCACACGTCTGAACTCCAGTC | Y-Shape 5′-strand |
| NO: 30 | | | |
| SEQ ID | duplex_3bp_21_5′ | 5Phos/CACAGATCGGAAGAGCACACGTCTGAACTCCAGTC | Y-Shape 5′-strand |
| NO: 31 | | | |
| SEQ ID | duplex_3bp_22_5′ | 5Phos/CCGAGATCGGAAGAGCACACGTCTGAACTCCAGTC | Y-Shape 5′-strand |
| NO: 32 | | | |
| SEQ ID | duplex_3bp_23_5′ | 5Phos/CGTAGATCGGAAGAGCACACGTCTGAACTCCAGTC | Y-Shape 5′-strand |
| NO: 33 | | | |
| SEQ ID | duplex_3bp_24_5′ | 5Phos/CTAAGATCGGAAGAGCACACGTCTGAACTCCAGTC | Y-Shape 5′-strand |
| NO: 34 | | | |
| SEQ ID | duplex_3bp_25_5′ | 5Phos/CAGAGATCGGAAGAGCACACGTCTGAACTCCAGTC | Y-Shape 5′-strand |
| NO: 35 | | | |
| SEQ ID | duplex_3bp_26_5′ | 5Phos/CCTAGATCGGAAGAGCACACGTCTGAACTCCAGTC | Y-Shape 5′-strand |
| NO: 36 | | | |
| SEQ ID | duplex_3bp_27_5′ | 5Phos/CGAAGATCGGAAGAGCACACGTCTGAACTCCAGTC | Y-Shape 5′-strand |
| NO: 37 | | | |
| SEQ ID | duplex_3bp_28_5 | 5Phos/CTCAGATCGGAAGAGCACACGTCTGAACTCCAGTC | Y-Shape 5′-strand |
| NO: 38 | | | |
| SEQ ID | duplex_3bp_29_5′ | 5Phos/CATAGATCGGAAGAGCACACGTCTGAACTCCAGTC | Y-Shape 5′-strand |
| NO: 39 | | | |
| SEQ ID | duplex_3bp_30_5′ | 5Phos/CCAAGATCGGAAGAGCACACGTCTGAACTCCAGTC | Y-Shape 5′-strand |
| NO: 40 | | | |
| SEQ ID | duplex_3bp_31_5′ | 5Phos/CGCAGATCGGAAGAGCACACGTCTGAACTCCAGTC | Y-Shape 5′-strand |
| NO: 41 | | | |
| SEQ ID | duplex_3bp_32_5′ | 5Phos/CTGAGATCGGAAGAGCACACGTCTGAACTCCAGTC | Y-Shape 5′-strand |
| NO: 42 | | | |
| SEQ ID | duplex_3bp_33_5′ | 5Phos/GAAAGATCGGAAGAGCACACGTCTGAACTCCAGTC | Y-Shape 5′-strand |
| NO: 43 | | | |
| SEQ ID | duplex_3bp_34_5′ | 5Phos/GCCAGATCGGAAGAGCACACGTCTGAACTCCAGTC | Y-Shape 5′-strand |
| NO: 44 | | | |
| SEQ ID | duplex_3bp_35_5′ | 5Phos/GGGAGATCGGAAGAGCACACGTCTGAACTCCAGTC | Y-Shape 5′-strand |
| NO: 45 | | | |
| SEQ ID | duplex_3bp_36_5′ | 5Phos/GTTAGATCGGAAGAGCACACGTCTGAACTCCAGTC | Y-Shape 5′-strand |
| NO: 46 | | | |
| SEQ ID | duplex_3bp_37_5′ | 5Phos/GACAGATCGGAAGAGCACACGTCTGAACTCCAGTC | Y-Shape 5′-strand |
| NO: 47 | | | |
| SEQ ID | duplex_3bp_38_5′ | 5Phos/GCGAGATCGGAAGAGCACACGTCTGAACTCCAGTC | Y-Shape 5′-strand |
| NO: 48 | | | |
| SEQ ID | duplex_3bp_39_5′ | 5Phos/GGTAGATCGGAAGAGCACACGTCTGAACTCCAGTC | Y-Shape 5′-strand |
| NO: 49 | | | |
| SEQ ID | duplex_3bp_40_5′ | 5Phos/GTAAGATCGGAAGAGCACACGTCTGAACTCCAGTC | Y-Shape 5′-strand |
| NO: 50 | | | |
| SEQ ID | duplex_3bp_41_5′ | 5Phos/GAGAGATCGGAAGAGCACACGTCTGAACTCCAGTC | Y-Shape 5′-strand |
| NO: 51 | | | |
| SEQ ID | duplex_3bp_42_5′ | 5Phos/GCTAGATCGGAAGAGCACACGTCTGAACTCCAGTC | Y-Shape 5′-strand |
| NO: 52 | | | |
| SEQ ID | duplex_3bp_43_5 | 5Phos/GGAAGATCGGAAGAGCACACGTCTGAACTCCAGTC | Y-Shape 5′-strand |
| NO: 53 | | | |
| SEQ ID | duplex_3bp_44_5′ | 5Phos/GTCAGATCGGAAGAGCACACGTCTGAACTCCAGTC | Y-Shape 5′-strand |
| NO: 54 | | | |
| SEQ ID | duplex_3bp_45_5′ | 5Phos/GATAGATCGGAAGAGCACACGTCTGAACTCCAGTC | Y-Shape 5′-strand |
| NO: 55 | | | |
| SEQ ID | duplex_3bp_46_5′ | 5Phos/GCAAGATCGGAAGAGCACACGTCTGAACTCCAGTC | Y-Shape 5′-strand |
| NO: 56 | | | |
| SEQ ID | duplex_3bp_47_5′ | 5Phos/GGCAGATCGGAAGAGCACACGTCTGAACTCCAGTC | Y-Shape 5′-strand |
| NO: 57 | | | |
| SEQ ID | duplex_3bp_48_5′ | 5Phos/GTGAGATCGGAAGAGCACACGTCTGAACTCCAGTC | Y-Shape 5′-strand |
| NO: 58 | | | |
| SEQ ID | duplex_3bp_49_5′ | 5Phos/CTAAAGATCGGAAGAGCACACGTCTGAACTCCAGTC | Y-Shape 5′-strand |
| NO: 59 | | | |
| SEQ ID | duplex_3bp_50_5′ | 5Phos/CTCCAGATCGGAAGAGCACACGTCTGAACTCCAGTC | Y-Shape 5′-strand |
| NO: 60 | | | |
| SEQ ID | duplex_3bp_51_5′ | 5Phos/CTGGAGATCGGAAGAGCACACGTCTGAACTCCAGTC | Y-Shape 5′-strand |
| NO: 61 | | | |
| SEQ ID | duplex_3bp_52_5′ | 5Phos/CTTTAGATCGGAAGAGCACACGTCTGAACTCCAGTC | Y-Shape 5′-strand |
| NO: 62 | | | |
| SEQ ID | duplex_3bp_53_5′ | 5Phos/CTACAGATCGGAAGAGCACACGTCTGAACTCCAGTC | Y-Shape 5′-strand |
| NO: 63 | | | |
| SEQ ID | duplex_3bp_54_5′ | 5Phos/CTCGAGATCGGAAGAGCACACGTCTGAACTCCAGTC | Y-Shape 5′-strand |
| NO: 64 | | | |
| SEQ ID | duplex_3bp_55_5′ | 5Phos/CTGTAGATCGGAAGAGCACACGTCTGAACTCCAGTC | Y-Shape 5′-strand |
| NO: 65 | | | |
| SEQ ID | duplex_3bp_56_5′ | 5Phos/CTTAAGATCGGAAGAGCACACGTCTGAACTCCAGTC | Y-Shape 5′-strand |
| NO: 66 | | | |
| SEQ ID | duplex_3bp_57_5′ | 5Phos/GTAGAGATCGGAAGAGCACACGTCTGAACTCCAGTC | Y-Shape 5′-strand |
| NO: 67 | | | |
| SEQ ID | duplex_3bp_58_5 | 5Phos/GTCTAGATCGGAAGAGCACACGTCTGAACTCCAGTC | Y-Shape 5′-strand |
| NO: 68 | | | |
| SEQ ID | duplex_3bp_59_5′ | 5Phos/GTGAAGATCGGAAGAGCACACGTCTGAACTCCAGTC | Y-Shape 5′-strand |
| NO: 69 | | | |
| SEQ ID | duplex_3bp_60_5′ | 5Phos/GTTCAGATCGGAAGAGCACACGTCTGAACTCCAGTC | Y-Shape 5′-strand |
| NO: 70 | | | |
| SEQ ID | duplex_3bp_61_5′ | 5Phos/GTATAGATCGGAAGAGCACACGTCTGAACTCCAGTC | Y-Shape 5′-strand |
| NO: 71 | | | |
| SEQ ID | duplex_3bp_62_5′ | 5Phos/GTCAAGATCGGAAGAGCACACGTCTGAACTCCAGTC | Y-Shape 5′-strand |
| NO: 72 | | | |
| SEQ ID | duplex_3bp_63_5′ | 5Phos/GTGCAGATCGGAAGAGCACACGTCTGAACTCCAGTC | Y-Shape 5′-strand |
| NO: 73 | | | |
| SEQ ID | duplex_3bp_64_5′ | 5Phos/GTTGAGATCGGAAGAGCACACGTCTGAACTCCAGTC | Y-Shape 5′-strand |
| NO: 74 | | | |
| SEQ ID | duplex_3bp_1_3′ | ACACTCTTTCCCTACACGACGCTCTTCCGATCTTTTG*T | Y-Shape 3′-strand |
| NO: 75 | | | |
| SEQ ID | duplex_3bp_2_3′ | ACACTCTTTCCCTACACGACGCTCTTCCGATCTGGTG*T | Y-Shape 3′-strand |
| NO: 76 | | | |
| SEQ ID | duplex_3bp_3_3′ | ACACTCTTTCCCTACACGACGCTCTTCCGATCTCCTG*T | Y-Shape 3′-strand |
| NO: 77 | | | |
| SEQ ID | duplex_3bp_4_3′ | ACACTCTTTCCCTACACGACGCTCTTCCGATCTAATG*T | Y-Shape 3′-strand |
| NO: 78 | | | |
| SEQ ID | duplex_3bp_5_3′ | ACACTCTTTCCCTACACGACGCTCTTCCGATCTGTTG*T | Y-Shape 3′-strand |
| NO: 79 | | | |
| SEQ ID | duplex_3bp_6_3′ | ACACTCTTTCCCTACACGACGCTCTTCCGATCTCGTG*T | Y-Shape 3′-strand |
| NO: 80 | | | |
| SEQ ID | duplex_3bp_7_3′ | ACACTCTTTCCCTACACGACGCTCTTCCGATCTACTG*T | Y-Shape 3′-strand |
| NO: 81 | | | |
| SEQ ID | duplex_3bp_8_3′ | ACACTCTTTCCCTACACGACGCTCTTCCGATCTTATG*T | Y-Shape 3′-strand |
| NO: 82 | | | |
| SEQ ID | duplex_3bp_9_3 | ACACTCTTTCCCTACACGACGCTCTTCCGATCTCTTC*T | Y-Shape 3′-strand |
| NO: 83 | | | |
| SEQ ID | duplex_3bp_10_3′ | ACACTCTTTCCCTACACGACGCTCTTCCGATCTAGTC*T | Y-Shape 3′-strand |
| NO: 84 | | | |
| SEQ ID | duplex_3bp_11_3′ | ACACTCTTTCCCTACACGACGCTCTTCCGATCTTCTC*T | Y-Shape 3′-strand |
| NO: 85 | | | |
| SEQ ID | duplex_3bp_12_3′ | ACACTCTTTCCCTACACGACGCTCTTCCGATCTGATC*T | Y-Shape 3′-strand |
| NO: 86 | | | |
| SEQ ID | duplex_3bp_13_3′ | ACACTCTTTCCCTACACGACGCTCTTCCGATCTATTC*T | Y-Shape 3′-strand |
| NO: 87 | | | |
| SEQ ID | duplex_3bp_14_3′ | ACACTCTTTCCCTACACGACGCTCTTCCGATCTTGTC*T | Y-Shape 3′-strand |
| NO: 88 | | | |
| SEQ ID | duplex_3bp_15_3′ | ACACTCTTTCCCTACACGACGCTCTTCCGATCTGCTC*T | Y-Shape 3′-strand |
| NO: 89 | | | |
| SEQ ID | duplex_3bp_16_3′ | ACACTCTTTCCCTACACGACGCTCTTCCGATCTCATC*T | Y-Shape 3′-strand |
| NO: 90 | | | |
| SEQ ID | duplex_3bp_17_3′ | ACACTCTTTCCCTACACGACGCTCTTCCGATCTTTG*T | Y-Shape 3′-strand |
| NO: 91 | | | |
| SEQ ID | duplex_3bp_18_3′ | ACACTCTTTCCCTACACGACGCTCTTCCGATCTGGG*T | Y-Shape 3′-strand |
| NO: 92 | | | |
| SEQ ID | duplex_3bp_19_3′ | ACACTCTTTCCCTACACGACGCTCTTCCGATCTCCG*T | Y-Shape 3′-strand |
| NO: 93 | | | |
| SEQ ID | duplex_3bp_20_3′ | ACACTCTTTCCCTACACGACGCTCTTCCGATCTAAG*T | Y-Shape 3′-strand |
| NO: 94 | | | |
| SEQ ID | duplex_3bp_21_3′ | ACACTCTTTCCCTACACGACGCTCTTCCGATCTGTG*T | Y-Shape 3′-strand |
| NO: 95 | | | |
| SEQ ID | duplex_3bp_22_3′ | ACACTCTTTCCCTACACGACGCTCTTCCGATCTCGG*T | Y-Shape 3′-strand |
| NO: 96 | | | |
| SEQ ID | duplex_3bp_23_3′ | ACACTCTTTCCCTACACGACGCTCTTCCGATCTACG*T | Y-Shape 3′-strand |
| NO: 97 | | | |
| SEQ ID | duplex_3bp_24_3 | ACACTCTTTCCCTACACGACGCTCTTCCGATCTTAG*T | Y-Shape 3′-strand |
| NO: 98 | | | |
| SEQ ID | duplex_3bp_25_3′ | ACACTCTTTCCCTACACGACGCTCTTCCGATCTCTG*T | Y-Shape 3′-strand |
| NO: 99 | | | |
| SEQ ID | duplex_3bp_26_3′ | ACACTCTTTCCCTACACGACGCTCTTCCGATCTAGG*T | Y-Shape 3′-strand |
| NO: 100 | | | |
| SEQ ID | duplex_3bp_27_3′ | ACACTCTTTCCCTACACGACGCTCTTCCGATCTTCG*T | Y-Shape 3′-strand |
| NO: 101 | | | |
| SEQ ID | duplex_3bp_28_3′ | ACACTCTTTCCCTACACGACGCTCTTCCGATCTGAG*T | Y-Shape 3′-strand |
| NO: 102 | | | |
| SEQ ID | duplex_3bp_29_3′ | ACACTCTTTCCCTACACGACGCTCTTCCGATCTATG*T | Y-Shape 3′-strand |
| NO: 103 | | | |
| SEQ ID | duplex_3bp_30_3′ | ACACTCTTTCCCTACACGACGCTCTTCCGATCTTGG*T | Y-Shape 3′-strand |
| NO: 104 | | | |
| SEQ ID | duplex_3bp_31_3′ | ACACTCTTTCCCTACACGACGCTCTTCCGATCTGCG*T | Y-Shape 3′-strand |
| NO: 105 | | | |
| SEQ ID | duplex_3bp_32_3′ | ACACTCTTTCCCTACACGACGCTCTTCCGATCTCAG*T | Y-Shape 3′-strand |
| NO: 106 | | | |
| SEQ ID | duplex_3bp_33_3′ | ACACTCTTTCCCTACACGACGCTCTTCCGATCTTTC*T | Y-Shape 3′-strand |
| NO: 107 | | | |
| SEQ ID | duplex_3bp_34_3′ | ACACTCTTTCCCTACACGACGCTCTTCCGATCTGGC*T | Y-Shape 3′-strand |
| NO: 108 | | | |
| SEQ ID | duplex_3bp_35_3′ | ACACTCTTTCCCTACACGACGCTCTTCCGATCTCCC*T | Y-Shape 3′-strand |
| NO: 109 | | | |
| SEQ ID | duplex_3bp_36_3′ | ACACTCTTTCCCTACACGACGCTCTTCCGATCTAAC*T | Y-Shape 3′-strand |
| NO: 110 | | | |
| SEQ ID | duplex_3bp_37_3′ | ACACTCTTTCCCTACACGACGCTCTTCCGATCTGTC*T | Y-Shape 3′-strand |
| NO: 111 | | | |
| SEQ ID | duplex_3bp_38_3′ | ACACTCTTTCCCTACACGACGCTCTTCCGATCTCGC*T | Y-Shape 3′-strand |
| NO: 112 | | | |
| SEQ ID | duplex_3bp_39_3 | ACACTCTTTCCCTACACGACGCTCTTCCGATCTACC*T | Y-Shape 3′-strand |
| NO: 113 | | | |
| SEQ ID | duplex_3bp_40_3′ | ACACTCTTTCCCTACACGACGCTCTTCCGATCTTAC*T | Y-Shape 3′-strand |
| NO: 114 | | | |
| SEQ ID | duplex_3bp_41_3′ | ACACTCTTTCCCTACACGACGCTCTTCCGATCTCTC*T | Y-Shape 3′-strand |
| NO: 115 | | | |
| SEQ ID | duplex_3bp_42_3′ | ACACTCTTTCCCTACACGACGCTCTTCCGATCTAGC*T | Y-Shape 3′-strand |
| NO: 116 | | | |
| SEQ ID | duplex_3bp_43_3′ | ACACTCTTTCCCTACACGACGCTCTTCCGATCTTCC*T | Y-Shape 3′-strand |
| NO: 117 | | | |
| SEQ ID | duplex_3bp_44_3′ | ACACTCTTTCCCTACACGACGCTCTTCCGATCTGAC*T | Y-Shape 3′-strand |
| NO: 118 | | | |
| SEQ ID | duplex_3bp_45_3′ | ACACTCTTTCCCTACACGACGCTCTTCCGATCTATC*T | Y-Shape 3′-strand |
| NO: 119 | | | |
| SEQ ID | duplex_3bp_46_3′ | ACACTCTTTCCCTACACGACGCTCTTCCGATCTTGC*T | Y-Shape 3′-strand |
| NO: 120 | | | |
| SEQ ID | duplex_3bp_47_3′ | ACACTCTTTCCCTACACGACGCTCTTCCGATCTGCC*T | Y-Shape 3′-strand |
| NO: 121 | | | |
| SEQ ID | duplex_3bp_48_3′ | ACACTCTTTCCCTACACGACGCTCTTCCGATCTCAC*T | Y-Shape 3′-strand |
| NO: 122 | | | |
| SEQ ID | duplex_3bp_49_3′ | ACACTCTTTCCCTACACGACGCTCTTCCGATCTTTAG*T | Y-Shape 3′-strand |
| NO: 123 | | | |
| SEQ ID | duplex_3bp_50_3′ | ACACTCTTTCCCTACACGACGCTCTTCCGATCTGGAG*T | Y-Shape 3′-strand |
| NO: 124 | | | |
| SEQ ID | duplex_3bp_51_3′ | ACACTCTTTCCCTACACGACGCTCTTCCGATCTCCAG*T | Y-Shape 3′-strand |
| NO: 125 | | | |
| SEQ ID | duplex_3bp_52_3′ | ACACTCTTTCCCTACACGACGCTCTTCCGATCTAAAG*T | Y-Shape 3′-strand |
| NO: 126 | | | |
| SEQ ID | duplex_3bp_53_3′ | ACACTCTTTCCCTACACGACGCTCTTCCGATCTGTAG*T | Y-Shape 3′-strand |
| NO: 127 | | | |
| SEQ ID | duplex_3bp_54_3 | ACACTCTTTCCCTACACGACGCTCTTCCGATCTCGAG*T | Y-Shape 3′-strand |
| NO: 128 | | | |
| SEQ ID | duplex_3bp_55_3′ | ACACTCTTTCCCTACACGACGCTCTTCCGATCTACAG*T | Y-Shape 3′-strand |
| NO: 129 | | | |
| SEQ ID | duplex_3bp_56_3′ | ACACTCTTTCCCTACACGACGCTCTTCCGATCTTAAG*T | Y-Shape 3′-strand |
| NO: 130 | | | |
| SEQ ID | duplex_3bp_57_3′ | ACACTCTTTCCCTACACGACGCTCTTCCGATCTCTAC*T | Y-Shape 3′-strand |
| NO: 131 | | | |
| SEQ ID | duplex_3bp_58_3′ | ACACTCTTTCCCTACACGACGCTCTTCCGATCTAGAC*T | Y-Shape 3′-strand |
| NO: 132 | | | |
| SEQ ID | duplex_3bp_59_3′ | ACACTCTTTCCCTACACGACGCTCTTCCGATCTTCAC*T | Y-Shape 3′-strand |
| NO: 133 | | | |
| SEQ ID | duplex_3bp_60_3′ | ACACTCTTTCCCTACACGACGCTCTTCCGATCTGAAC*T | Y-Shape 3′-strand |
| NO: 134 | | | |
| SEQ ID | duplex_3bp_61_3′ | ACACTCTTTCCCTACACGACGCTCTTCCGATCTATAC*T | Y-Shape 3′-strand |
| NO: 135 | | | |
| SEQ ID | duplex_3bp_62_3′ | ACACTCTTTCCCTACACGACGCTCTTCCGATCTTGAC*T | Y-Shape 3′-strand |
| NO: 136 | | | |
| SEQ ID | duplex_3bp_63_3′ | ACACTCTTTCCCTACACGACGCTCTTCCGATCTGCAC*T | Y-Shape 3′-strand |
| NO: 137 | | | |
| SEQ ID | duplex_3bp_64_3′ | ACACTCTTTCCCTACACGACGCTCTTCCGATCTCAAC*T | Y-Shape 3′-strand |
| NO: 138 | | | |
| SEQ ID | duplex_3bp_1 | /5Phos/CAAAAGATCGGAAGAGCACACGTCTGAACTCCAGTC | Looped |
| NO: 139 | | /ideoxyU/ACACTCTTTCCCTACACGACGCTCTTCCGATCTTTT | |
| | | G*T | |
| SEQ ID | duplex_3bp_2 | /5Phos/CACCAGATCGGAAGAGCACACGTCTGAACTCCAGTC | Looped |
| NO: 140 | | /ideoxyU/ACACTCTTTCCCTACACGACGCTCTTCCGATCTGGT | |
| | | G*T | |
| SEQ ID | duplex_3bp_3 | /5Phos/CAGGAGATCGGAAGAGCACACGTCTGAACTCCAGT | Looped |
| NO: 141 | | C/ideoxyU/ACACTCTTTCCCTACACGACGCTCTTCCGATCTCC | |
| | | TG*T | |
| SEQ ID | duplex_3bp_4 | /5Phos/CATTAGATCGGAAGAGCACACGTCTGAACTCCAGTC | Looped |
| NO: 142 | | /ideoxyU/ACACTCTTTCCCTACACGACGCTCTTCCGATCTAAT | |
| | | G*T | |
| SEQ ID | duplex_3bp_5 | /5Phos/CAACAGATCGGAAGAGCACACGTCTGAACTCCAGTC | Looped |
| NO: 143 | | /ideoxyU/ACACTCTTTCCCTACACGACGCTCTTCCGATCTGTT | |
| | | G*T | |
| SEQ ID | duplex_3bp_6 | /5Phos/CACGAGATCGGAAGAGCACACGTCTGAACTCCAGT | Looped |
| NO: 144 | | C/ideoxyU/ACACTCTTTCCCTACACGACGCTCTTCCGATCTCG | |
| | | TG*T | |
| SEQ ID | duplex_3bp_7 | /5Phos/CAGTAGATCGGAAGAGCACACGTCTGAACTCCAGTC | Looped |
| NO: 145 | | /ideoxyU/ACACTCTTTCCCTACACGACGCTCTTCCGATCTACT | |
| | | G*T | |
| SEQ ID | duplex_3bp_8 | /5Phos/CATAAGATCGGAAGAGCACACGTCTGAACTCCAGTC | Looped |
| NO: 146 | | /ideoxyU/ACACTCTTTCCCTACACGACGCTCTTCCGATCTTAT | |
| | | G*T | |
| SEQ ID | duplex_3bp_9 | /5Phos/GAAGAGATCGGAAGAGCACACGTCTGAACTCCAGT | Looped |
| NO: 147 | | C/ideoxyU/ACACTCTTTCCCTACACGACGCTCTTCCGATCTCT | |
| | | TC*T | |
| SEQ ID | duplex_3bp_10 | /5Phos/GACTAGATCGGAAGAGCACACGTCTGAACTCCAGTC | Looped |
| NO: 148 | | /ideoxyU/ACACTCTTTCCCTACACGACGCTCTTCCGATCTAGT | |
| | | C*T | |
| SEQ ID | duplex_3bp_11 | /5Phos/GAGAAGATCGGAAGAGCACACGTCTGAACTCCAGT | Looped |
| NO: 149 | | C/ideoxyU/ACACTCTTTCCCTACACGACGCTCTTCCGATCTTC | |
| | | TC*T | |
| SEQ ID | duplex_3bp_12 | /5Phos/GATCAGATCGGAAGAGCACACGTCTGAACTCCAGTC | Looped |
| NO: 150 | | /ideoxyU/ACACTCTTTCCCTACACGACGCTCTTCCGATCTGAT | |
| | | C*T | |
| SEQ ID | duplex_3bp_13 | /5Phos/GAATAGATCGGAAGAGCACACGTCTGAACTCCAGT | Looped |
| NO: 151 | | C/ideoxyU/ACACTCTTTCCCTACACGACGCTCTTCCGATCTAT | |
| | | TC*T | |
| SEQ ID | duplex_3bp_14 | /5Phos/GACAAGATCGGAAGAGCACACGTCTGAACTCCAGT | Looped |
| NO: 152 | | C/ideoxyU/ACACTCTTTCCCTACACGACGCTCTTCCGATCTTG | |
| | | TC*T | |
| SEQ ID | duplex_3bp_15 | /5Phos/GAGCAGATCGGAAGAGCACACGTCTGAACTCCAGT | Looped |
| NO: 153 | | C/ideoxyU/ACACTCTTTCCCTACACGACGCTCTTCCGATCTGC | |
| | | TC*T | |
| SEQ ID | duplex_3bp_16 | /5Phos/GATGAGATCGGAAGAGCACACGTCTGAACTCCAGT | Looped |
| NO: 154 | | C/ideoxyU/ACACTCTTTCCCTACACGACGCTCTTCCGATCTCA | |
| | | TC*T | |
| SEQ ID | duplex_3bp_17 | /5Phos/CAAAGATCGGAAGAGCACACGTCTGAACTCCAGTC/ | Looped |
| NO: 155 | | ideoxyU/ACACTCTTTCCCTACACGACGCTCTTCCGATCTTTG* | |
| | | T | |
| SEQ ID | duplex_3bp_18 | /5Phos/CCCAGATCGGAAGAGCACACGTCTGAACTCCAGTC/i | Looped |
| NO: 156 | | deoxyU/ACACTCTTTCCCTACACGACGCTCTTCCGATCTGGG* | |
| | | T | |
| SEQ ID | duplex_3bp_19 | /5Phos/CGGAGATCGGAAGAGCACACGTCTGAACTCCAGTC/ | Looped |
| NO: 157 | | ideoxyU/ACACTCTTTCCCTACACGACGCTCTTCCGATCTCCG | |
| | | *T | |
| SEQ ID | duplex_3bp_20 | /5Phos/CTTAGATCGGAAGAGCACACGTCTGAACTCCAGTC/i | Looped |
| NO: 158 | | deoxyU/ACACTCTTTCCCTACACGACGCTCTTCCGATCTAAG* | |
| | | T | |
| SEQ ID | duplex_3bp_21 | /5Phos/CACAGATCGGAAGAGCACACGTCTGAACTCCAGTC/i | Looped |
| NO: 159 | | deoxyU/ACACTCTTTCCCTACACGACGCTCTTCCGATCTGTG* | |
| | | T | |
| SEQ ID | duplex_3bp_22 | /5Phos/CCGAGATCGGAAGAGCACACGTCTGAACTCCAGTC/ | Looped |
| NO: 160 | | ideoxyU/ACACTCTTTCCCTACACGACGCTCTTCCGATCTCGG | |
| | | *T | |
| SEQ ID | duplex_3bp_23 | /5Phos/CGTAGATCGGAAGAGCACACGTCTGAACTCCAGTC/ | Looped |
| NO: 161 | | ideoxyU/ACACTCTTTCCCTACACGACGCTCTTCCGATCTACG* | |
| | | T | |
| SEQ ID | duplex_3bp_24 | /5Phos/CTAAGATCGGAAGAGCACACGTCTGAACTCCAGTC/ | Looped |
| NO: 162 | | deioxyU/ACACTCTTTCCCTACACGACGCTCTTCCGATCTTAG* | |
| | | T | |
| SEQ ID | duplex_3bp_25 | /5Phos/CAGAGATCGGAAGAGCACACGTCTGAACTCCAGTC/ | Looped |
| NO: 163 | | ideoxyU/ACACTCTTTCCCTACACGACGCTCTTCCGATCTCTG* | |
| | | T | |
| SEQ ID | duplex_3bp_26 | /5Phos/CCTAGATCGGAAGAGCACACGTCTGAACTCCAGTC/ | Looped |
| NO: 164 | | ideoxyU/ACACTCTTTCCCTACACGACGCTCTTCCGATCTAGG* | |
| | | T | |
| SEQ ID | duplex_3bp_27 | /5Phos/CGAAGATCGGAAGAGCACACGTCTGAACTCCAGTC/ | Looped |
| NO: 165 | | ideoxyU/ACACTCTTTCCCTACACGACGCTCTTCCGATCTTCG* | |
| | | T | |
| SEQ ID | duplex_3bp_28 | /5Phos/CTCAGATCGGAAGAGCACACGTCTGAACTCCAGTC/ | Looped |
| NO: 166 | | ideoxyU/ACACTCTTTCCCTACACGACGCTCTTCCGATCTGAG* | |
| | | T | |
| SEQ ID | duplex_3bp_29 | /5Phos/CATAGATCGGAAGAGCACACGTCTGAACTCCAGTC/ | Looped |
| NO: 167 | | dieoxyU/ACACTCTTTCCCTACACGACGCTCTTCCGATCTATG* | |
| | | T | |
| SEQ ID | duplex_3bp_30 | /5Phos/CCAAGATCGGAAGAGCACACGTCTGAACTCCAGTC/ | Looped |
| NO: 168 | | ideoxyU/ACACTCTTTCCCTACACGACGCTCTTCCGATCTTGG* | |
| | | T | |
| SEQ ID | duplex_3bp_31 | /5Phos/CGCAGATCGGAAGAGCACACGTCTGAACTCCAGTC/ | Looped |
| NO: 169 | | ideoxyU/ACACTCTTTCCCTACACGACGCTCTTCCGATCTGCG | |
| | | *T | |
| SEQ ID | duplex_3bp_32 | /5Phos/CTGAGATCGGAAGAGCACACGTCTGAACTCCAGTC/ | Looped |
| NO: 170 | | ideoxyU/ACACTCTTTCCCTACACGACGCTCTTCCGATCTCAG* | |
| | | T | |
| SEQ ID | duplex_3bp_33 | /5Phos/GAAAGATCGGAAGAGCACACGTCTGAACTCCAGTC/ | Looped |
| NO: 171 | | ideoxyU/ACACTCTTTCCCTACACGACGCTCTTCCGATCTTTC* | |
| | | T | |
| SEQ ID | duplex_3bp_34 | /5Phos/GCCAGATCGGAAGAGCACACGTCTGAACTCCAGTC/ | Looped |
| NO: 172 | | ideoxyU/ACACTCTTTCCCTACACGACGCTCTTCCGATCTGGC | |
| | | *T | |
| SEQ ID | duplex_3bp_35 | /5Phos/GGGAGATCGGAAGAGCACACGTCTGAACTCCAGTC | Looped |
| NO: 173 | | /ideoxyU/ACACTCTTTCCCTACACGACGCTCTTCCGATCTCCC | |
| | | *T | |
| SEQ ID | duplex_3bp_36 | /5Phos/GTTAGATCGGAAGAGCACACGTCTGAACTCCAGTC/ | Looped |
| NO: 174 | | ideoxyU/ACACTCTTTCCCTACACGACGCTCTTCCGATCTAAC* | |
| | | T | |
| SEQ ID | duplex_3bp_37 | /5Phos/GACAGATCGGAAGAGCACACGTCTGAACTCCAGTC/ | Looped |
| NO: 175 | | ideoxyU/ACACTCTTTCCCTACACGACGCTCTTCCGATCTGTC* | |
| | | T | |
| SEQ ID | duplex_3bp_38 | /5Phos/GCGAGATCGGAAGAGCACACGTCTGAACTCCAGTC/ | Looped |
| NO: 176 | | ideoxyU/ACACTCTTTCCCTACACGACGCTCTTCCGATCTCGC | |
| | | *T | |
| SEQ ID | duplex_3bp_39 | /5Phos/GGTAGATCGGAAGAGCACACGTCTGAACTCCAGTC/ | Looped |
| NO: 177 | | ideoxyU/ACACTCTTTCCCTACACGACGCTCTTCCGATCTACC* | |
| | | T | |
| SEQ ID | duplex_3bp_40 | /5Phos/GTAAGATCGGAAGAGCACACGTCTGAACTCCAGTC/ | Looped |
| NO: 178 | | ideoxyU/ACACTCTTTCCCTACACGACGCTCTTCCGATCTTAC* | |
| | | T | |
| SEQ ID | duplex_3bp_41 | /5Phos/GAGAGATCGGAAGAGCACACGTCTGAACTCCAGTC/ | Looped |
| NO: 179 | | ideoxyU/ACACTCTTTCCCTACACGACGCTCTTCCGATCTCTC* | |
| | | T | |
| SEQ ID | duplex_3bp_42 | /5Phos/GCTAGATCGGAAGAGCACACGTCTGAACTCCAGTC/ | Looped |
| NO: 180 | | ideoxyU/ACACTCTTTCCCTACACGACGCTCTTCCGATCTAGC* | |
| | | T | |
| SEQ ID | duplex_3bp_43 | /5Phos/GGAAGATCGGAAGAGCACACGTCTGAACTCCAGTC/ | Looped |
| NO: 181 | | ideoxyU/ACACTCTTTCCCTACACGACGCTCTTCCGATCTTCC* | |
| | | T | |
| SEQ ID | duplex_3bp_44 | /5Phos/GTCAGATCGGAAGAGCACACGTCTGAACTCCAGTC/ | Looped |
| NO: 182 | | ideoxyU/ACACTCTTTCCCTACACGACGCTCTTCCGATCTGAC* | |
| | | T | |
| SEQ ID | duplex_3bp_45 | /5Phos/GATAGATCGGAAGAGCACACGTCTGAACTCCAGTC/ | Looped |
| NO: 183 | | ideoxyU/ACACTCTTTCCCTACACGACGCTCTTCCGATCTATC* | |
| | | T | |
| SEQ ID | duplex_3bp_46 | /5Phos/GCAAGATCGGAAGAGCACACGTCTGAACTCCAGTC/ | Looped |
| NO: 184 | | ideoxyU/ACACTCTTTCCCTACACGACGCTCTTCCGATCTTGC* | |
| | | T | |
| SEQ ID | duplex_3bp_47 | /5Phos/GGCAGATCGGAAGAGCACACGTCTGAACTCCAGTC/ | Looped |
| NO: 185 | | ideoxyU/ACACTCTTTCCCTACACGACGCTCTTCCGATCTGCC | |
| | | *T | |
| SEQ ID | duplex_3bp_48 | /5Phos/GTGAGATCGGAAGAGCACACGTCTGAACTCCAGTC/ | Looped |
| NO: 186 | | ideoxyU/ACACTCTTTCCCTACACGACGCTCTTCCGATCTCAC* | |
| | | T | |
| SEQ ID | duplex_3bp_49 | /5Phos/CTAAAGATCGGAAGAGCACACGTCTGAACTCCAGTC | Looped |
| NO: 187 | | /ideoxyU/ACACTCTTTCCCTACACGACGCTCTTCCGATCTTTA | |
| | | G*T | |
| SEQ ID | duplex_3bp_50 | /5Phos/CTCCAGATCGGAAGAGCACACGTCTGAACTCCAGTC | Looped |
| NO: 188 | | /ideoxyU/ACACTCTTTCCCTACACGACGCTCTTCCGATCTGG | |
| | | AG*T | |
| SEQ ID | duplex_3bp_51 | /5Phos/CTGGAGATCGGAAGAGCACACGTCTGAACTCCAGT | Looped |
| NO: 189 | | C/ideoxyU/ACACTCTTTCCCTACACGACGCTCTTCCGATCTCC | |
| | | AG*T | |
| SEQ ID | duplex_3bp_52 | /5Phos/CTTTAGATCGGAAGAGCACACGTCTGAACTCCAGTC | Looped |
| NO: 190 | | /ideoxyU/ACACTCTTTCCCTACACGACGCTCTTCCGATCTAAA | |
| | | G*T | |
| SEQ ID | duplex_3bp_53 | /5Phos/CTACAGATCGGAAGAGCACACGTCTGAACTCCAGTC | Looped |
| NO: 191 | | /ideoxyU/ACACTCTTTCCCTACACGACGCTCTTCCGATCTGTA | |
| | | G*T | |
| SEQ ID | duplex_3bp_54 | /5Phos/CTCGAGATCGGAAGAGCACACGTCTGAACTCCAGTC | Looped |
| NO: 192 | | /ideoxyU/ACACTCTTTCCCTACACGACGCTCTTCCGATCTCGA | |
| | | G*T | |
| SEQ ID | duplex_3bp_55 | /5Phos/CTGTAGATCGGAAGAGCACACGTCTGAACTCCAGTC | Looped |
| NO: 193 | | /ideoxyU/ACACTCTTTCCCTACACGACGCTCTTCCGATCTACA | |
| | | G*T | |
| SEQ ID | duplex_3bp_56 | /5Phos/CTTAAGATCGGAAGAGCACACGTCTGAACTCCAGTC | Looped |
| NO: 194 | | /ideoxyU/ACACTCTTTCCCTACACGACGCTCTTCCGATCTTAA | |
| | | G*T | |
| SEQ ID | duplex_3bp_57 | /5Phos/GTAGAGATCGGAAGAGCACACGTCTGAACTCCAGT | Looped |
| NO: 195 | | C/ideoxyU/ACACTCTTTCCCTACACGACGCTCTTCCGATCTCT | |
| | | AC*T | |
| SEQ ID | duplex_3bp_58 | /5Phos/GTCTAGATCGGAAGAGCACACGTCTGAACTCCAGTC | Looped |
| NO: 196 | | /ideoxyU/ACACTCTTTCCCTACACGACGCTCTTCCGATCTAGA | |
| | | C*T | |
| SEQ ID | duplex_3bp_59 | /5Phos/GTGAAGATCGGAAGAGCACACGTCTGAACTCCAGT | Looped |
| NO: 197 | | C/ideoxyU/ACACTCTTTCCCTACACGACGCTCTTCCGATCTTC | |
| | | AC*T | |
| SEQ ID | duplex_3bp_60 | /5Phos/GTTCAGATCGGAAGAGCACACGTCTGAACTCCAGTC | Looped |
| NO: 198 | | /ideoxyU/ACACTCTTTCCCTACACGACGCTCTTCCGATCTGAA | |
| | | C*T | |
| SEQ ID | duplex_3bp_61 | /5Phos/GTATAGATCGGAAGAGCACACGTCTGAACTCCAGTC | Looped |
| NO: 199 | | /ideoxyU/ACACTCTTTCCCTACACGACGCTCTTCCGATCTATA | |
| | | C*T | |
| SEQ ID | duplex_3bp_62 | /5Phos/GTCAAGATCGGAAGAGCACACGTCTGAACTCCAGTC | Looped |
| NO: 200 | | /ideoxyU/ACACTCTTTCCCTACACGACGCTCTTCCGATCTTGA | |
| | | C*T | |
| SEQ ID | duplex_3bp_63 | /5Phos/GTGCAGATCGGAAGAGCACACGTCTGAACTCCAGT | Looped |
| NO: 201 | | C/ideoxyU/ACACTCTTTCCCTACACGACGCTCTTCCGATCTGC | |
| | | AC*T | |
| SEQ ID | duplex_3bp_64 | /5Phos/GTTGAGATCGGAAGAGCACACGTCTGAACTCCAGTC | Looped |
| NO: 202 | | /ideoxyU/ACACTCTTTCCCTACACGACGCTCTTCCGATCTCAA | |
| | | C*T | |

Oligonucleotide sequences are shown 5′-3′. * = phosphorothioate; n = 2a, c, g, or t; s = 2c or g; w = a or t; ideoxyU = internal uracil.

### Exemplary Embodiments

Exemplary embodiments provided in accordance with the presently disclosed subject matter include, but are not limited to, the claims and the following embodiments.

A1. A method for preparing nucleic acid sequences for sequencing:
a. providing at least one barcoded hairpin adapter, wherein the barcoded hairpin adapter contains a cleavable linkage;
b. cleaving the cleavable linkage with a cleaving agent to create a cleaved barcoded adapter, wherein the cleaved barcoded adapter comprises a double stranded region and two single stranded tails;
c. providing at least one sample of randomly fragmented double stranded nucleic acid target;
d. ligating the cleaved barcoded adapter to each end of the target to generate an adapter-target-adapter; and
e. amplifying the adaptor-target-adapter with two or more amplification primers, wherein the two or more amplification primers are complementary to the single stranded tails.
   A2. The method of embodiment A1, wherein the barcoded hairpin adapter contains a barcode region from 2-6 nucleotide base pairs.
   A3. The method of embodiment A1, wherein the barcoded hairpin adapters form a complex mix of 1 to 16 different adapters.
   A4. The method of embodiment A1, wherein the barcoded hairpin adapters form a complex mix of 1 to 64 different adapters.
   A5. The method of embodiment of A1, wherein the barcoded hairpin adapters form a complex mix of 1 to 256 different adapters.
   A6. The method of embodiment A1, wherein the barcoded hairpin adapters form a complex mix of 1 to 1024 different adapters.
   A7. The method of embodiment of A1, wherein the barcoded hairpin adapters form a complex mix of 1 to 4096 different adapters.
   A8. A method for preparing nucleic acid sequences for sequencing:
a. providing at least one barcoded hairpin adapter, wherein the barcoded hairpin adapter contains a cleavable linkage;
b. providing at least one sample of randomly fragmented double stranded nucleic acid target;
c. combining the barcoded hairpin adapter, target, cleavage agent, and ligase into a single reaction tube to generate an adapter-target-adapter;
d. amplifying the adaptor-target-adapter with two or more amplification primers.
   A9. A method for preparing nucleic acid sequences for sequencing;
a. providing a sample of randomly fragmented double stranded nucleic acid target;
b. ligating a barcoded hairpin adapter to each end of the target to generate an adapter-target-adapter;
c. amplifying the adapter-target-adapter with two or more amplification primers.
   A10. A method of sequencing DNA comprising:
a. independently sequencing first and second strands of dsDNA to obtain corresponding first and second sequences; and
b. combining the first and second sequences to generate a consensus sequence of the dsDNA.
   A11. A double stranded oligonucleotide comprising:
a double stranded stem region having a unique molecular identifier (UMI); and
a single stranded loop region.
   A12. The double stranded oligonucleotide of claim 11, wherein the unique molecular identifier is at least 2 base pairs.
   B1. A method of sequencing DNA comprising:
a) Ligating a partially double stranded unique barcoded adapter to a target double stranded DNA, to form an adapter-target-adapter complex;
b) Amplifying each strand of the adapter-target-adapter complex to produce a plurality of amplified first strand adapter-target-adapter complexes and a plurality of amplified second strand adapter-target-adapter complexes;
c) independently sequencing the amplified adapter-target adapter complexes to form a plurality of first strand reads and a plurality of second strand reads;
d) combining at least one first strand read to at least one second strand read and generating a plurality of consensus sequences; and
e) analyzing at least one sequence form the consensus sequence and generating an error corrected sequence read of the first and second sequences to generate a consensus sequence of the target double stranded DNA.
   B2. The method of claim 1, wherein the partially double stranded unique barcoded adapter is Y-shaped or looped.
   B3. The method of claim 1, wherein the partially double stranded unique barcoded adapter comprises a unique sequence, wherein the unique sequence comprises 2 to 6 nucleotide bases.
   B4. The method of claim 3, wherein the partially double stranded unique barcoded adapter contains a unique sequence, wherein the unique sequence is 2 nucleotide bases.
   B5. The method of claim 1, wherein the partially double stranded unique barcoded adapters consist of 64 unique adapter molecules.
   B6. The method of claim 1, wherein the partially double stranded unique barcoded adapters consist of 16 unique barcoded adapter molecules.
   C1. A plurality of duplexed barcoded adapters comprising: SEQ ID NO:1, SEQ ID NO:2, SEQ ID NO:3, SEQ ID NO:4, or a combination thereof.
   D1. A plurality of duplexed barcoded adapters comprising: SEQ ID NO:5, SEQ ID NO:6, SEQ ID NO:7, SEQ ID NO:8, or a combination thereof.
   E1. A looped barcoded adapters comprising SEQ ID NO: 8.
   F1. A looped barcoded adapter comprising SEQ ID NO: 9.

What is claimed is:

1. A method of sequencing DNA comprising:
f) Ligating a partially double stranded unique barcoded adapter to a target double stranded DNA, to form an adapter-target-adapter complex;
g) Amplifying each strand of the adapter-target-adapter complex to produce a plurality of amplified first strand adapter-target-adapter complexes and a plurality of amplified second strand adapter-target-adapter complexes;
h) independently sequencing the amplified adapter-target adapter complexes to form a plurality of first strand reads and a plurality of second strand reads;
i) combining at least one first strand read to at least one second strand read and generating a plurality of consensus sequences; and
j) analyzing at least one sequence form the consensus sequence and generating an error corrected sequence read of the first and second sequences to generate a consensus sequence of the target double stranded DNA.

2. The method of claim 1, wherein the partially double stranded unique barcoded adapter is Y-shaped or looped.

3. The method of claim 1, wherein the partially double stranded unique barcoded adapter comprises a unique sequence, wherein the unique sequence comprises 2 to 6 nucleotide bases.

4. The method of claim 3, wherein the partially double stranded unique barcoded adapter contains a unique sequence, wherein the unique sequence is 2 nucleotide bases.

5. The method of claim 1, wherein the partially double stranded unique barcoded adapters consist of 64 unique adapter molecules.

6. The method of claim 1, wherein the partially double stranded unique barcoded adapters consist of 16 unique barcoded adapter molecules.

7. A plurality of duplexed barcoded adapters comprising: SEQ ID NO:1, SEQ ID NO:2, SEQ ID NO:3, SEQ ID NO:4, or a combination thereof.

8. A plurality of duplexed barcoded adapters comprising: SEQ ID NO:5, SEQ ID NO:6, SEQ ID NO:7, SEQ ID NO:8, or a combination thereof.


SEQUENCE LISTING (202 sequences)

SEQ ID NO: 1 - DNA, 36 bp, Artificial Sequence
  Synthetic nucleic acid
  misc_feature (1)..(1): 5Phos
  misc_feature (1)..(1): s is c or g
  misc_feature (2)..(2): w is a or t
  misc_feature (3)..(4): n is a, c, g, or t
swnnagatcggaagagcacacgtctgaactccagtc

SEQ ID NO: 2 - DNA, 35 bp, Artificial Sequence
  Synthetic nucleic acid
  misc_feature (1)..(1): 5Phos
  misc_feature (1)..(1): s is c or g
  misc_feature (2)..(3): n is a, c, g, or t
snnagatcggaagagcacacgtctgaactccagtc

SEQ ID NO: 3 - DNA, 38 bp, Artificial Sequence
  Synthetic nucleic acid
  misc_feature (34)..(35): n is a,c, g, or t
  misc_feature (36)..(36): w is a or t
  misc_feature (37)..(37): s is c or g
  misc_feature (38)..(38): Phosphorthioate linkage
acactctttccctacacgacgctcttccgatctnnwst

SEQ ID NO: 4 - DNA, 37 bp, Artificial Sequence
  Synthetic nucleic acid
  misc_feature (34)..(35): n is a, c, g, or t
  misc_feature (36)..(36): s is c or g
  misc_feature (37)..(37): Phosphorothioate linkage
acactctttccctacacgacgctcttccgatctnnst

SEQ ID NO: 5 - DNA, 35 bp, Artificial Sequence
  Synthetic nucleic acid
  misc_feature (1)..(1): s is c or g
  misc_feature (1)..(1): 5Phos
  misc_feature (1)..(1): s is c or g
  misc_feature (2)..(2): w is a or t
  misc_feature (3)..(3): n is a, c, g, or t
swnagatcggaagagcacacgtctgaactccagtc

SEQ ID NO: 6 - DNA, 34 bp, Artificial Sequence
  Synthetic nucleic acid
  misc_feature (1)..(1): 5Phos
  misc_feature (1)..(1): s is c or g
  misc_feature (2)..(2): n is a, c, g, or t
snagatcggaagagcacacgtctgaactccagtc

SEQ ID NO: 7 - DNA, 37 bp, Artificial Sequence
  Synthetic nucleic acid
  misc_feature (34)..(34): n is a, c, g, or t
  misc_feature (35)..(35): w is a or t
  misc_feature (36)..(36): s is c or g
  misc_feature (37)..(37): Phosphorothioate linkage
acactctttccctacacgacgctcttccgatctnwst

SEQ ID NO: 8 - DNA, 36 bp, Artificial Sequence
  Synthetic nucleic acid
  misc_feature (34)..(34): n is a, c, g, or t
  misc_feature (35)..(35): s is c or g
  misc_feature (36)..(36): Phosphorothioate linkage
acactctttccctacacgacgctcttccgatctnst

SEQ ID NO: 9 - DNA, 75 bp, Artificial Sequence
  Synthetic nucleic acid
  misc_feature (1)..(1): 5Phos
  misc_feature (1)..(1): s is c or g
  misc_feature (2)..(2): w is a or t
  misc_feature (3)..(4): n is a, c, g, or t
  misc_feature (46)..(46): deoxy uracil
  misc_feature (71)..(72): n is a, c, g, or t
  misc_feature (73)..(73): w is a or t
  misc_feature (74)..(74): s is c or g
  misc_feature (75)..(75): Phosphorotioate linkage
swnnagatcggaagagcacacgtctgaactccagtcuacactctttccctacacgacgctcttccgatctnnwst

SEQ ID NO: 10 - DNA, 73 bp, Artificial Sequence
  Synthetic nucleic acid
  misc_feature (1)..(1): 5Phos
  misc_feature (1)..(1): s is c or g
  misc_feature (2)..(3): n is a, c, g, or t
  misc_feature (45)..(45): deoxy uracil
  misc_feature (69)..(70): n is a, c, g, or t
  misc_feature (71)..(71): s is c or g
  misc_feature (72)..(72): Phosphorothioate linkage
snnagatcggaagagcacacgtctgaactccagtcuacactctttccctacacgacgctcttccgatctnnst

SEQ ID NO: 11 - DNA, 36 bp, Artificial Sequence
  Synthetic nucleic acid
  misc_feature (1)..(1): 5Phos
caaaagatcggaagagcacacgtctgaactccagtc

SEQ ID NO: 12 - DNA, 36 bp, Artificial Sequence
  Synthetic nucleic acid
  misc_feature (1)..(1): 5Phos
caccagatcggaagagcacacgtctgaactccagtc

SEQ ID NO: 13 - DNA, 36 bp, Artificial Sequence
  Synthetic nucleic acid
  misc_feature (1)..(1): 5Phos
caggagatcggaagagcacacgtctgaactccagtc

SEQ ID NO: 14 - DNA, 36 bp, Artificial Sequence
  Synthetic nucleic acid
  misc_feature (1)..(1): 5Phos
cattagatcggaagagcacacgtctgaactccagtc

SEQ ID NO: 15 - DNA, 36 bp, Artificial Sequence
  Synthetic nucleic acid
  misc_feature (1)..(1): 5Phos
caacagatcggaagagcacacgtctgaactccagtc

SEQ ID NO: 16 - DNA, 36 bp, Artificial Sequence
  Synthetic nucleic acid
  misc_feature (1)..(1): 5Phos
cacgagatcggaagagcacacgtctgaactccagtc

SEQ ID NO: 17 - DNA, 36 bp, Artificial Sequence
  Synthetic nucleic acid
  misc_feature (1)..(1): 5Phos
cagtagatcggaagagcacacgtctgaactccagtc

SEQ ID NO: 18 - DNA, 36 bp, Artificial Sequence
  Synthetic nucleic acid
  misc_feature (1)..(1): 5Phos
cataagatcggaagagcacacgtctgaactccagtc

SEQ ID NO: 19 - DNA, 36 bp, Artificial Sequence
  Synthetic nucleic acid
  misc_feature (1)..(1): 5Phos
gaagagatcggaagagcacacgtctgaactccagtc

SEQ ID NO: 20 - DNA, 36 bp, Artificial Sequence
  Synthetic nucleic acid
  misc_feature (1)..(1): 5Phos
gactagatcggaagagcacacgtctgaactccagtc

SEQ ID NO: 21 - DNA, 36 bp, Artificial Sequence
  Synthetic nucleic acid
  misc_feature (1)..(1): 5Phos
gagaagatcggaagagcacacgtctgaactccagtc

SEQ ID NO: 22 - DNA, 36 bp, Artificial Sequence
  Synthetic nucleic acid
  misc_feature (1)..(1): 5Phos
gatcagatcggaagagcacacgtctgaactccagtc

SEQ ID NO: 23 - DNA, 36 bp, Artificial Sequence
  Synthetic nucleic acid
  misc_feature (1)..(1): 5Phos
gaatagatcggaagagcacacgtctgaactccagtc

SEQ ID NO: 24 - DNA, 36 bp, Artificial Sequence
  Synthetic nucleic acid
  misc_feature (1)..(1): 5Phos
gacaagatcggaagagcacacgtctgaactccagtc

SEQ ID NO: 25 - DNA, 36 bp, Artificial Sequence
  Synthetic nucleic acid
  misc_feature (1)..(1): 5Phos
gagcagatcggaagagcacacgtctgaactccagtc

SEQ ID NO: 26 - DNA, 36 bp, Artificial Sequence
  Synthetic nucleic acid
  misc_feature (1)..(1): 5Phos
gatgagatcggaagagcacacgtctgaactccagtc

SEQ ID NO: 27 - DNA, 35 bp, Artificial Sequence
  Synthetic nucleic acid
  misc_feature (1)..(1): 5Phos
caaagatcggaagagcacacgtctgaactccagtc

SEQ ID NO: 28 - DNA, 35 bp, Artificial Sequence
  Synthetic nucleic acid
  misc_feature (1)..(1): 5Phos
cccagatcggaagagcacacgtctgaactccagtc

SEQ ID NO: 29 - DNA, 35 bp, Artificial Sequence
  Synthetic nucleic acid
  misc_feature (1)..(1): 5Phos
cggagatcggaagagcacacgtctgaactccagtc

SEQ ID NO: 30 - DNA, 35 bp, Artificial Sequence
  Synthetic nucleic acid
  misc_feature (1)..(1): 5Phos
cttagatcggaagagcacacgtctgaactccagtc

SEQ ID NO: 31 - DNA, 35 bp, Artificial Sequence
  Synthetic nucleic acid
  misc_feature (1)..(1): 5Phos
cacagatcggaagagcacacgtctgaactccagtc

SEQ ID NO: 32 - DNA, 35 bp, Artificial Sequence
  Synthetic nucleic acid
  misc_feature (1)..(1): 5Phos
ccgagatcggaagagcacacgtctgaactccagtc

SEQ ID NO: 33 - DNA, 35 bp, Artificial Sequence
  Synthetic nucleic acid
  misc_feature (1)..(1)
cgtagatcggaagagcacacgtctgaactccagtc

SEQ ID NO: 34 - DNA, 35 bp, Artificial Sequence
  Synthetic nucleic acid
  misc_feature (1)..(1): 5Phos
ctaagatcggaagagcacacgtctgaactccagtc

SEQ ID NO: 35 - DNA, 35 bp, Artificial Sequence
  Synthetic nucleic acid
  misc_feature (1)..(1): 5Phos
cagagatcggaagagcacacgtctgaactccagtc

SEQ ID NO: 36 - DNA, 35 bp, Artificial Sequence
  Synthetic nucleic acid
  misc_feature (1)..(1): 5Phos
cctagatcggaagagcacacgtctgaactccagtc

SEQ ID NO: 37 - DNA, 35 bp, Artificial Sequence
  Synthetic nucleic acid
  misc_feature (1)..(1): 5Phos
cgaagatcggaagagcacacgtctgaactccagtc

SEQ ID NO: 38 - DNA, 35 bp, Artificial Sequence
  Synthetic nucleic acid
  misc_feature (1)..(1): 5Phos
ctcagatcggaagagcacacgtctgaactccagtc

SEQ ID NO: 39 - DNA, 35 bp, Artificial Sequence
  Synthetic nucleic acid
  misc_feature (1)..(1): 5Phos
catagatcggaagagcacacgtctgaactccagtc

SEQ ID NO: 40 - DNA, 35 bp, Artificial Sequence
  Synthetic nucleic acid
  misc_feature (1)..(1): 5Phos
ccaagatcggaagagcacacgtctgaactccagtc

SEQ ID NO: 41 - DNA, 35 bp, Artificial Sequence
  Synthetic nucleic acid
  misc_feature (1)..(1): 5Phos
cgcagatcggaagagcacacgtctgaactccagtc

SEQ ID NO: 42 - DNA, 35 bp, Artificial Sequence
  Synthetic nucleic acid
  misc_feature (1)..(1): 5Phos
ctgagatcggaagagcacacgtctgaactccagtc

SEQ ID NO: 43 - DNA, 35 bp, Artificial Sequence
  Synthetic nucleic acid
  misc_feature (1)..(1): 5Phos
gaaagatcggaagagcacacgtctgaactccagtc

SEQ ID NO: 44 - DNA, 35 bp, Artificial Sequence
  Synthetic nucleic acid
  misc_feature (1)..(1): 5Phos
gccagatcggaagagcacacgtctgaactccagtc

SEQ ID NO: 45 - DNA, 35 bp, Artificial Sequence
  Synthetic nucleic acid
  misc_feature (1)..(1): 5Phos
gggagatcggaagagcacacgtctgaactccagtc

SEQ ID NO: 46 - DNA, 31 bp, Artificial Sequence
  Synthetic nucleic acid
  misc_feature (1)..(1): 5Phos
gttagatcggaagagcacacgtctgaactcc

SEQ ID NO: 47 - DNA, 35 bp, Artificial Sequence
  Synthetic nucleic acid
  misc_feature (1)..(1): 5Phos
gacagatcggaagagcacacgtctgaactccagtc

SEQ ID NO: 48 - DNA, 35 bp, Artificial Sequence
  Synthetic nucleic acid
  misc_feature (1)..(1): 5Phos
gcgagatcggaagagcacacgtctgaactccagtc

SEQ ID NO: 49 - DNA, 35 bp, Artificial Sequence
  Synthetic nucleic acid
  misc_feature (1)..(1): 5Phos
ggtagatcggaagagcacacgtctgaactccagtc

SEQ ID NO: 50 - DNA, 35 bp, Artificial Sequence
  Synthetic nucleic acid
  misc_feature (1)..(1): 5Phos
gtaagatcggaagagcacacgtctgaactccagtc

SEQ ID NO: 51 - DNA, 35 bp, Artificial Sequence
  Synthetic nucleic acid
  misc_feature (1)..(1): 5Phos
gagagatcggaagagcacacgtctgaactccagtc

SEQ ID NO: 52 - DNA, 35 bp, Artificial Sequence
  Synthetic nucleic acid
  misc_feature (1)..(1): 5Phos
gctagatcggaagagcacacgtctgaactccagtc

SEQ ID NO: 53 - DNA, 35 bp, Artificial Sequence
  Synthetic nucleic acid
  misc_feature (1)..(1): 5Phos
ggaagatcggaagagcacacgtctgaactccagtc

SEQ ID NO: 54 - DNA, 35 bp, Artificial Sequence
  Synthetic nucleic acid
  misc_feature (1)..(1): 5Phos
gtcagatcggaagagcacacgtctgaactccagtc

SEQ ID NO: 55 - DNA, 35 bp, Artificial Sequence
  Synthetic nucleic acid
  misc_feature (1)..(1): 5Phos
gatagatcggaagagcacacgtctgaactccagtc

SEQ ID NO: 56 - DNA, 35 bp, Artificial Sequence
  Synthetic nucleic acid
  misc_feature (1)..(1): 5Phos
gcaagatcggaagagcacacgtctgaactccagtc

SEQ ID NO: 57 - DNA, 35 bp, Artificial Sequence
  Synthetic nucleic acid
  misc_feature (1)..(1): 5Phos
ggcagatcggaagagcacacgtctgaactccagtc

SEQ ID NO: 58 - DNA, 35 bp, Artificial Sequence
  Synthetic nucleic acid
  misc_feature (1)..(1): 5Phos
gtgagatcggaagagcacacgtctgaactccagtc

SEQ ID NO: 59 - DNA, 36 bp, Artificial Sequence
  Synthetic nucleic acid
  misc_feature (1)..(1): 5Phos
ctaaagatcggaagagcacacgtctgaactccagtc

SEQ ID NO: 60 - DNA, 36 bp, Artificial Sequence
  Synthetic nucleic acid
  misc_feature (1)..(1): 5Phos
ctccagatcggaagagcacacgtctgaactccagtc

SEQ ID NO: 61 - DNA, 36 bp, Artificial Sequence
  Synthetic nucleic acid
  misc_feature (1)..(1): 5Phos
ctggagatcggaagagcacacgtctgaactccagtc

SEQ ID NO: 62 - DNA, 36 bp, Artificial Sequence
  Synthetic nucleic acid
  misc_feature (1)..(1): 5Phos
ctttagatcggaagagcacacgtctgaactccagtc

SEQ ID NO: 63 - DNA, 36 bp, Artificial Sequence
  Synthetic nucleic acid
  misc_feature (1)..(1): 5Phos
ctacagatcggaagagcacacgtctgaactccagtc

SEQ ID NO: 64 - DNA, 36 bp, Artificial Sequence
  Synthetic nucleic acid
  misc_feature (1)..(1): 5Phos
ctcgagatcggaagagcacacgtctgaactccagtc

SEQ ID NO: 65 - DNA, 36 bp, Artificial Sequence
  Synthetic nucleic acid
  misc_feature (1)..(1): 5Phos
ctgtagatcggaagagcacacgtctgaactccagtc

SEQ ID NO: 66 - DNA, 36 bp, Artificial Sequence
  Synthetic nucleic acid
  misc_feature (1)..(1): 5Phos
cttaagatcggaagagcacacgtctgaactccagtc

SEQ ID NO: 67 - DNA, 36 bp, Artificial Sequence
  Synthetic nucleic acid
  misc_feature (1)..(1): 5Phos
gtagagatcggaagagcacacgtctgaactccagtc

SEQ ID NO: 68 - DNA, 36 bp, Artificial Sequence
  Synthetic nucleic acid
  misc_feature (1)..(1): 5Phos
gtctagatcggaagagcacacgtctgaactccagtc

SEQ ID NO: 69 - DNA, 36 bp, Artificial Sequence
  Synthetic nucleic acid
  misc_feature (1)..(1): 5Phos
gtgaagatcggaagagcacacgtctgaactccagtc

SEQ ID NO: 70 - DNA, 36 bp, Artificial Sequence
  Synthetic nucleic acid
  misc_feature (1)..(1): 5Phos
gttcagatcggaagagcacacgtctgaactccagtc

SEQ ID NO: 71 - DNA, 36 bp, Artificial Sequence
  Synthetic nucleic acid
  misc_feature (1)..(1): 5Phos
gtatagatcggaagagcacacgtctgaactccagtc

SEQ ID NO: 72 - DNA, 36 bp, Artificial Sequence
  Synthetic nucleic acid
  misc_feature (1)..(1): 5Phos
gtcaagatcggaagagcacacgtctgaactccagtc

SEQ ID NO: 73 - DNA, 36 bp, Artificial Sequence
  Synthetic nucleic acid
  misc_feature (1)..(1): 5Phos
gtgcagatcggaagagcacacgtctgaactccagtc

SEQ ID NO: 74 - DNA, 36 bp, Artificial Sequence
  Synthetic nucleic acid
  misc_feature (1)..(1): 5Phos
gttgagatcggaagagcacacgtctgaactccagtc

SEQ ID NO: 75 - DNA, 38 bp, Artificial Sequence
  Synthetic nucleic acid
  misc_feature (38)..(38): PS
acactctttccctacacgacgctcttccgatcttttgt

SEQ ID NO: 76 - DNA, 38 bp, Artificial Sequence
  Synthetic nucleic acid
  misc_feature (38)..(38): PS
acactctttccctacacgacgctcttccgatctggtgt

SEQ ID NO: 77 - DNA, 38 bp, Artificial Sequence
  Synthetic nucleic acid
  misc_feature (38)..(38): PS
acactctttccctacacgacgctcttccgatctcctgt

SEQ ID NO: 78 - DNA, 38 bp, Artificial Sequence
  Synthetic nucleic acid
  misc_feature (38)..(38): PS
acactctttccctacacgacgctcttccgatctaatgt

SEQ ID NO: 79 - DNA, 38 bp, Artificial Sequence
  Synthetic nucleic acid
  misc_feature (38)..(38): PS
acactctttccctacacgacgctcttccgatctgttgt

SEQ ID NO: 80 - DNA, 38 bp, Artificial Sequence
  Synthetic nucleic acid
  misc_feature (38)..(38): PS
acactctttccctacacgacgctcttccgatctcgtgt

SEQ ID NO: 81 - DNA, 38 bp, Artificial Sequence
  Synthetic nucleic acid
  misc_feature (38)..(38): PS
acactctttccctacacgacgctcttccgatctactgt

SEQ ID NO: 82 - DNA, 38 bp, Artificial Sequence
  Synthetic nucleic acid
  misc_feature (38)..(38): PS
acactctttccctacacgacgctcttccgatcttatgt

SEQ ID NO: 83 - DNA, 38 bp, Artificial Sequence
  Synthetic nucleic acid
  misc_feature (38)..(38): PS
acactctttccctacacgacgctcttccgatctcttct

SEQ ID NO: 84 - DNA, 38 bp, Artificial Sequence
  Synthetic nucleic acid
  misc_feature (38)..(38): ps
acactctttccctacacgacgctcttccgatctagtct

SEQ ID NO: 85 - DNA, 38 bp, Artificial Sequence
  Synthetic nucleic acid
  misc_feature (38)..(38): PS
acactctttccctacacgacgctcttccgatcttctct

SEQ ID NO: 86 - DNA, 38 bp, Artificial Sequence
  Synthetic nucleic acid
  misc_feature (38)..(38): PS
acactctttccctacacgacgctcttccgatctgatct

SEQ ID NO: 87 - DNA, 38 bp, Artificial Sequence
  Synthetic nucleic acid
  misc_feature (38)..(38): PS
acactctttccctacacgacgctcttccgatctattct

SEQ ID NO: 88 - DNA, 38 bp, Artificial Sequence
  Synthetic nucleic acid
  misc_feature (38)..(38): PS
acactctttccctacacgacgctcttccgatcttgtct

SEQ ID NO: 89 - DNA, 38 bp, Artificial Sequence
  Synthetic nucleic acid
  misc_feature (38)..(38): PS
acactctttccctacacgacgctcttccgatctgctct

SEQ ID NO: 90 - DNA, 38 bp, Artificial Sequence
  Synthetic nucleic acid
  misc_feature (38)..(38): PS
acactctttccctacacgacgctcttccgatctcatct

SEQ ID NO: 91 - DNA, 37 bp, Artificial Sequence
  Synthetic nucleic acid
  misc_feature (37)..(37): PS
acactctttccctacacgacgctcttccgatctttgt

SEQ ID NO: 92 - DNA, 37 bp, Artificial Sequence
  Synthetic nucleic acid
  misc_feature (37)..(37): PS
acactctttccctacacgacgctcttccgatctgggt

SEQ ID NO: 93 - DNA, 37 bp, Artificial Sequence
  Synthetic nucleic acid
  misc_feature (37)..(37): PS
acactctttccctacacgacgctcttccgatctccgt

SEQ ID NO: 94 - DNA, 37 bp, Artificial Sequence
  Synthetic nucleic acid
  misc_feature (37)..(37): PS
acactctttccctacacgacgctcttccgatctaagt

SEQ ID NO: 95 - DNA, 37 bp, Artificial Sequence
  Synthetic nucleic acid
  misc_feature (37)..(37): PS
acactctttccctacacgacgctcttccgatctgtgt

SEQ ID NO: 96 - DNA, 37 bp, Artificial Sequence
  Synthetic nucleic acid
  misc_feature (37)..(37): PS
acactctttccctacacgacgctcttccgatctcggt

SEQ ID NO: 97 - DNA, 37 bp, Artificial Sequence
  Synthetic nucleic acid
  misc_feature (37)..(37): PS
acactctttccctacacgacgctcttccgatctacgt

SEQ ID NO: 98 - DNA, 37 bp, Artificial Sequence
  Synthetic nucleic acid
  misc_feature (37)..(37): PS
acactctttccctacacgacgctcttccgatcttagt

SEQ ID NO: 99 - DNA, 37 bp, Artificial Sequence
  Synthetic nucleic acid
  misc_feature (37)..(37): PS
acactctttccctacacgacgctcttccgatctctgt

SEQ ID NO: 100 - DNA, 37 bp, Artificial Sequence
  Synthetic nucleic acid
  misc_feature (37)..(37): PS
acactctttccctacacgacgctcttccgatctaggt

SEQ ID NO: 101 - DNA, 37 bp, Artificial Sequence
  Synthetic nucleic acid
  misc_feature (37)..(37): PS
acactctttccctacacgacgctcttccgatcttcgt

SEQ ID NO: 102 - DNA, 37 bp, Artificial Sequence
  Synthetic nucleic acid
  misc_feature (37)..(37): PS
acactctttccctacacgacgctcttccgatctgagt

SEQ ID NO: 103 - DNA, 37 bp, Artificial Sequence
  Synthetic nucleic acid
  misc_feature (37)..(37): PS
acactctttccctacacgacgctcttccgatctatgt

SEQ ID NO: 104 - DNA, 37 bp, Artificial Sequence
  Synthetic nucleic acid
  misc_feature (37)..(37): PS
acactctttccctacacgacgctcttccgatcttggt

SEQ ID NO: 105 - DNA, 37 bp, Artificial Sequence
  Synthetic nucleic acid
  misc_feature (37)..(37): PS
acactctttccctacacgacgctcttccgatctgcgt

SEQ ID NO: 106 - DNA, 37 bp, Artificial Sequence
  Synthetic nucleic acid
  misc_feature (37)..(37): PS
acactctttccctacacgacgctcttccgatctcagt

SEQ ID NO: 107 - DNA, 37 bp, Artificial Sequence
  Synthetic nucleic acid
  misc_feature (37)..(37): PS
acactctttccctacacgacgctcttccgatctttct

SEQ ID NO: 108 - DNA, 37 bp, Artificial Sequence
  Synthetic nucleic acid
  misc_feature (37)..(37): PS
acactctttccctacacgacgctcttccgatctggct

SEQ ID NO: 109 - DNA, 37 bp, Artificial Sequence
  Synthetic nucleic acid
  misc_feature (37)..(37): PS
acactctttccctacacgacgctcttccgatctccct

SEQ ID NO: 110 - DNA, 37 bp, Artificial Sequence
  Synthetic nucleic acid
  misc_feature (37)..(37): PS
acactctttccctacacgacgctcttccgatctaact

SEQ ID NO: 111 - DNA, 37 bp, Artificial Sequence
  Synthetic nucleic acid
  misc_feature (37)..(37): PS
acactctttccctacacgacgctcttccgatctgtct

SEQ ID NO: 112 - DNA, 37 bp, Artificial Sequence
  Synthetic nucleic acid
  misc_feature (37)..(37): PS
acactctttccctacacgacgctcttccgatctcgct

SEQ ID NO: 113 - DNA, 37 bp, Artificial Sequence
  Synthetic nucleic acid
  misc_feature (37)..(37): PS
acactctttccctacacgacgctcttccgatctacct

SEQ ID NO: 114 - DNA, 37 bp, Artificial Sequence
  Synthetic nucleic acid
  misc_feature (37)..(37): PS
acactctttccctacacgacgctcttccgatcttact

SEQ ID NO: 115 - DNA, 37 bp, Artificial Sequence
  Synthetic nucleic acid
  misc_feature (37)..(37): PS
acactctttccctacacgacgctcttccgatctctct

SEQ ID NO: 116 - DNA, 37 bp, Artificial Sequence
  Synthetic nucleic acid
  misc_feature (37)..(37): PS
acactctttccctacacgacgctcttccgatctagct

SEQ ID NO: 117 - DNA, 37 bp, Artificial Sequence
  Synthetic nucleic acid
  misc_feature (37)..(37): PS
acactctttccctacacgacgctcttccgatcttcct

SEQ ID NO: 118 - DNA, 37 bp, Artificial Sequence
  Synthetic nucleic acid
  misc_feature (37)..(37): PS
acactctttccctacacgacgctcttccgatctgact

SEQ ID NO: 119 - DNA, 37 bp, Artificial Sequence
  Synthetic nucleic acid
  misc_feature (37)..(37): PS
acactctttccctacacgacgctcttccgatctatct

SEQ ID NO: 120 - DNA, 37 bp, Artificial Sequence
  Synthetic nucleic acid
  misc_feature (37)..(37): PS
acactctttccctacacgacgctcttccgatcttgct

SEQ ID NO: 121 - DNA, 37 bp, Artificial Sequence
  Synthetic nucleic acid
  misc_feature (37)..(37): PS
acactctttccctacacgacgctcttccgatctgcct

SEQ ID NO: 122 - DNA, 37 bp, Artificial Sequence
  Synthetic nucleic acid
  misc_feature (37)..(37): PS
acactctttccctacacgacgctcttccgatctcact

SEQ ID NO: 123 - DNA, 38 bp, Artificial Sequence
  Synthetic nucleic acid
  misc_feature (38)..(38): ps
acactctttccctacacgacgctcttccgatctttagt

SEQ ID NO: 124 - DNA, 38 bp, Artificial Sequence
  Synthetic nucleic acid
  misc_feature (38)..(38): PS
acactctttccctacacgacgctcttccgatctggagt

SEQ ID NO: 125 - DNA, 38 bp, Artificial Sequence
  Synthetic nucleic acid
  misc_feature (38)..(38): PS
acactctttccctacacgacgctcttccgatctccagt

SEQ ID NO: 126 - DNA, 38 bp, Artificial Sequence
  Synthetic nucleic acid
  misc_feature (38)..(38): PS
acactctttccctacacgacgctcttccgatctaaagt

SEQ ID NO: 127 - DNA, 38 bp, Artificial Sequence
  Synthetic nucleic acid
  misc_feature (38)..(38): PS
acactctttccctacacgacgctcttccgatctgtagt

SEQ ID NO: 128 - DNA, 38 bp, Artificial Sequence
  Synthetic nucleic acid
  misc_feature (38)..(38): PS
acactctttccctacacgacgctcttccgatctcgagt

SEQ ID NO: 129 - DNA, 38 bp, Artificial Sequence
  Synthetic nucleic acid
  misc_feature (38)..(38): PS
acactctttccctacacgacgctcttccgatctacagt

SEQ ID NO: 130 - DNA, 38 bp, Artificial Sequence
  Synthetic nucleic acid
  misc_feature (38)..(38): PS
acactctttccctacacgacgctcttccgatcttaagt

SEQ ID NO: 131 - DNA, 38 bp, Artificial Sequence
  Synthetic nucleic acid
  misc_feature (38)..(38): PS
acactctttccctacacgacgctcttccgatctctact

SEQ ID NO: 132 - DNA, 38 bp, Artificial Sequence
  Synthetic nucleic acid
  misc_feature (38)..(38): PS
acactctttccctacacgacgctcttccgatctagact

SEQ ID NO: 133 - DNA, 38 bp, Artificial Sequence
  Synthetic nucleic acid
  misc_feature (38)..(38): PS
acactctttccctacacgacgctcttccgatcttcact

SEQ ID NO: 134 - DNA, 38 bp, Artificial Sequence
  Synthetic nucleic acid
  misc_feature (38)..(38): PS
acactctttccctacacgacgctcttccgatctgaact

SEQ ID NO: 135 - DNA, 38 bp, Artificial Sequence
  Synthetic nucleic acid
  misc_feature (38)..(38): PS
acactctttccctacacgacgctcttccgatctatact

SEQ ID NO: 136 - DNA, 38 bp, Artificial Sequence
  Synthetic nucleic acid
  misc_feature (38)..(38): PS
acactctttccctacacgacgctcttccgatcttgact

SEQ ID NO: 137 - DNA, 38 bp, Artificial Sequence
  Synthetic nucleic acid
  misc_feature (38)..(38): PS
acactctttccctacacgacgctcttccgatctgcact

SEQ ID NO: 138 - DNA, 38 bp, Artificial Sequence
  Synthetic nucleic acid
  misc_feature (38)..(38): PS
acactctttccctacacgacgctcttccgatctcaact

SEQ ID NO: 139 - DNA, 75 bp, Artificial Sequence
  Synthetic nucleic acid
  misc_feature (1)..(1): 5Phos
  misc_feature (46)..(46): Deoxy uracil
  misc_feature (75)..(75): PS
caaaagatcggaagagcacacgtctgaactccagtcuacactctttccctacacgacgctcttccgatcttttgt

SEQ ID NO: 140 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
caccagatcggaagagcacacgtctgaactccagtcuacactctttccctacacgacgctcttccgatctggtgt

SEQ ID NO: 141 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
caggagatcggaagagcacacgtctgaactccagtcuacactctttccctacacgacgctcttccgatctcctgt

SEQ ID NO: 142 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
cattagatcggaagagcacacgtctgaactccagtcuacactctttccctacacgacgctcttccgatctaatgt

SEQ ID NO: 143 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
caacagatcggaagagcacacgtctgaactccagtcuacactctttccctacacgacgctcttccgatctgttgt

SEQ ID NO: 144 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
cacgagatcggaagagcacacgtctgaactccagtcuacactctttccctacacgacgctcttccgatctcgtgt

SEQ ID NO: 145 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
cagtagatcggaagagcacacgtctgaactccagtcuacactctttccctacacgacgctcttccgatctactgt

SEQ ID NO: 146 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
cataagatcggaagagcacacgtctgaactccagtcuacactctttccctacacgacgctcttccgatcttatgt

SEQ ID NO: 147 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gaagagatcggaagagcacacgtctgaactccagtcuacactctttccctacacgacgctcttccgatctcttct

SEQ ID NO: 148 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gactagatcggaagagcacacgtctgaactccagtcuacactctttccctacacgacgctcttccgatctagtct

SEQ ID NO: 149 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gagaagatcggaagagcacacgtctgaactccagtcuacactctttccctacacgacgctcttccgatcttctct

SEQ ID NO: 150 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gatcagatcggaagagcacacgtctgaactccagtcuacactctttccctacacgacgctcttccgatctgatct

SEQ ID NO: 151 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gaatagatcggaagagcacacgtctgaactccagtcuacactctttccctacacgacgctcttccgatctattct

SEQ ID NO: 152 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gacaagatcggaagagcacacgtctgaactccagtcuacactctttccctacacgacgctcttccgatcttgtct

SEQ ID NO: 153 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gagcagatcggaagagcacacgtctgaactccagtcuacactctttccctacacgacgctcttccgatctgctct

SEQ ID NO: 154 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gatgagatcggaagagcacacgtctgaactccagtcuacactctttccctacacgacgctcttccgatctcatct

SEQ ID NO: 155 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
caaagatcggaagagcacacgtctgaactccagtcuacactctttccctacacgacgctcttccgatctttgt

SEQ ID NO: 156 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
cccagatcggaagagcacacgtctgaactccagtcuacactctttccctacacgacgctcttccgatctgggt

SEQ ID NO: 157 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
cggagatcggaagagcacacgtctgaactccagtcuacactctttccctacacgacgctcttccgatctccgt

SEQ ID NO: 158 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
cttagatcggaagagcacacgtctgaactccagtcuacactctttccctacacgacgctcttccgatctaagt

SEQ ID NO: 159 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
cacagatcggaagagcacacgtctgaactccagtcuacactctttccctacacgacgctcttccgatctgtgt

SEQ ID NO: 160 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
ccgagatcggaagagcacacgtctgaactccagtcuacactctttccctacacgacgctcttccgatctcggt

SEQ ID NO: 161 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
cgtagatcggaagagcacacgtctgaactccagtcuacactctttccctacacgacgctcttccgatctacgt

SEQ ID NO: 162 - DNA, 73 bp, Artificial Sequence
  Synthetic nucleic acid
  misc_feature (1)..(1): 5Phos
  misc_feature (45)..(5): Deoxy Uracil
  misc_feature (73)..(73): PS
ctaagatcggaagagcacacgtctgaactccagtcuacactctttccctacacgacgctcttccgatcttagt

SEQ ID NO: 163 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
cagagatcggaagagcacacgtctgaactccagtcuacactctttccctacacgacgctcttccgatctctgt

SEQ ID NO: 164 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
cctagatcggaagagcacacgtctgaactccagtcuacactctttccctacacgacgctcttccgatctaggt

SEQ ID NO: 165 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
cgaagatcggaagagcacacgtctgaactccagtcuacactctttccctacacgacgctcttccgatcttcgt

SEQ ID NO: 166 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
ctcagatcggaagagcacacgtctgaactccagtcuacactctttccctacacgacgctcttccgatctgagt

SEQ ID NO: 167 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
catagatcggaagagcacacgtctgaactccagtcuacactctttccctacacgacgctcttccgatctatgt

SEQ ID NO: 168 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
ccaagatcggaagagcacacgtctgaactccagtcuacactctttccctacacgacgctcttccgatcttggt

SEQ ID NO: 169 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
cgcagatcggaagagcacacgtctgaactccagtcuacactctttccctacacgacgctcttccgatctgcgt

SEQ ID NO: 170 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
ctgagatcggaagagcacacgtctgaactccagtcuacactctttccctacacgacgctcttccgatctcagt

SEQ ID NO: 171 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gaaagatcggaagagcacacgtctgaactccagtcuacactctttccctacacgacgctcttccgatctttct

SEQ ID NO: 172 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gccagatcggaagagcacacgtctgaactccagtcuacactctttccctacacgacgctcttccgatctggct

SEQ ID NO: 173 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gggagatcggaagagcacacgtctgaactccagtcuacactctttccctacacgacgctcttccgatctccct

SEQ ID NO: 174 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gttagatcggaagagcacacgtctgaactccagtcuacactctttccctacacgacgctcttccgatctaact

SEQ ID NO: 175 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gacagatcggaagagcacacgtctgaactccagtcuacactctttccctacacgacgctcttccgatctgtct

SEQ ID NO: 176 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gcgagatcggaagagcacacgtctgaactccagtcuacactctttccctacacgacgctcttccgatctcgct

SEQ ID NO: 177 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
ggtagatcggaagagcacacgtctgaactccagtcuacactctttccctacacgacgctcttccgatctacct

SEQ ID NO: 178 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gtaagatcggaagagcacacgtctgaactccagtcuacactctttccctacacgacgctcttccgatcttact

SEQ ID NO: 179 - DNA, 73 bp, Artificial Sequence
  Synthetic nucleic acid
  misc_feature (1)..(1): 5Phos
  misc_feature (45)..(5): Deoxy Uracil
  misc_feature (73)..(73): PS
gagagatcggaagagcacacgtctgaactccagtcuacactctttccctacacgacgctcttccgatctctct

SEQ ID NO: 180 - DNA, 73 bp, Artificial Sequence
  Synthetic nucleic acid
  misc_feature (1)..(1): 5Phos
  misc_feature (43)..(43): Deoxy Uracil
  misc_feature (73)..(73): PS
gctagatcggaagagcacacgtctgaactccagtcuacactctttccctacacgacgctcttccgatctagct

SEQ ID NO: 181 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
ggaagatcggaagagcacacgtctgaactccagtcuacactctttccctacacgacgctcttccgatcttcct

SEQ ID NO: 182 - DNA, 73 bp, Artificial Sequence
  Synthetic nucleic acid
  misc_feature (1)..(1): 5Phos
  misc_feature (45)..(5): Deoxy Uracil
  misc_feature (73)..(73): PS
gtcagatcggaagagcacacgtctgaactccagtcuacactctttccctacacgacgctcttccgatctgact

SEQ ID NO: 183 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gatagatcggaagagcacacgtctgaactccagtcuacactctttccctacacgacgctcttccgatctatct

SEQ ID NO: 184 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gcaagatcggaagagcacacgtctgaactccagtcuacactctttccctacacgacgctcttccgatcttgct

SEQ ID NO: 185 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
ggcagatcggaagagcacacgtctgaactccagtcuacactctttccctacacgacgctcttccgatctgcct

SEQ ID NO: 186 - DNA, 73 bp, Artificial Sequence
  Synthetic nucleic acid
  misc_feature (1)..(1): 5Phos
  misc_feature (45)..(45): Deoxy Uracil
  misc_feature (73)..(73): PS
gtgagatcggaagagcacacgtctgaactccagtcuacactctttccctacacgacgctcttccgatctcact

SEQ ID NO: 187 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
ctaaagatcggaagagcacacgtctgaactccagtcuacactctttccctacacgacgctcttccgatctttagt

SEQ ID NO: 188 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
ctccagatcggaagagcacacgtctgaactccagtcuacactctttccctacacgacgctcttccgatctggagt

SEQ ID NO: 189 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
ctggagatcggaagagcacacgtctgaactccagtcuacactctttccctacacgacgctcttccgatctccagt

SEQ ID NO: 190 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
ctttagatcggaagagcacacgtctgaactccagtcuacactctttccctacacgacgctcttccgatctaaagt

SEQ ID NO: 191 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
ctacagatcggaagagcacacgtctgaactccagtcuacactctttccctacacgacgctcttccgatctgtagt

SEQ ID NO: 192 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
ctcgagatcggaagagcacacgtctgaactccagtcuacactctttccctacacgacgctcttccgatctcgagt

SEQ ID NO: 193 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
ctgtagatcggaagagcacacgtctgaactccagtcuacactctttccctacacgacgctcttccgatctacagt

SEQ ID NO: 194 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
cttaagatcggaagagcacacgtctgaactccagtcuacactctttccctacacgacgctcttccgatcttaagt

SEQ ID NO: 195 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gtagagatcggaagagcacacgtctgaactccagtcuacactctttccctacacgacgctcttccgatctctact

SEQ ID NO: 196 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gtctagatcggaagagcacacgtctgaactccagtcuacactctttccctacacgacgctcttccgatctagact

SEQ ID NO: 197 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gtgaagatcggaagagcacacgtctgaactccagtcuacactctttccctacacgacgctcttccgatcttcact

SEQ ID NO: 198 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gttcagatcggaagagcacacgtctgaactccagtcuacactctttccctacacgacgctcttccgatctgaact

SEQ ID NO: 199 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gtatagatcggaagagcacacgtctgaactccagtcuacactctttccctacacgacgctcttccgatctatact

SEQ ID NO: 200 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gtcaagatcggaagagcacacgtctgaactccagtcuacactctttccctacacgacgctcttccgatcttgact

SEQ ID NO: 201 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gtgcagatcggaagagcacacgtctgaactccagtcuacactctttccctacacgacgctcttccgatctgcact

SEQ ID NO: 202 - DNA, 75 bp, Artificial Sequence
  Synthetic nucleic acid
  misc_feature (1)..(1): 5Phos
  misc_feature (46)..(46): Deoxy Uracil
  misc_feature (75)..(75): PS
gttgagatcggaagagcacacgtctgaactccagtcuacactctttccctacacgacgctcttccgatctcaact